(19) United States
(12) Patent Application Publication
(10) Pub. No.: US 2023/0127499 A1
(43) Pub. Date: Apr. 27, 2023

(54) PEPTIDES AND ADJUVANTS FOR AUGMENTATION OF FIBROBLAST THERAPY FOR CORONAVIRUS

(71) Applicant: FIGENE, LLC, Houston, TX (US)

(72) Inventors: Pete O'HEERON, Houston, TX (US); Thomas ICHIM, San Diego, CA (US)

(21) Appl. No.: 17/905,961

(22) PCT Filed: Mar. 2, 2021
(86) PCT No.: PCT/US21/20457

§ 371 Date: Sep. 9, 2022

Related U.S. Application Data
(60) Provisional application No. 63/002,134, filed on Mar. 30, 2020.
(60) Provisional application No. 63/003,742, filed on Apr. 1, 2020.

(51) Int. Cl. A61K 35/33; A61K 38/17; A61K 45/06; A61K 31/4706; A61P 29/00; A61P 11/00; A61K 38/16; A61K 31/7088; A61K 31/4745; A61K 31/7125; A61K 31/7052; A61K 31/05; A61K 31/4178

(52) U.S. Cl. CPC A61K 35/33; A61K 38/1774; A61K 45/06; A61K 31/4706; A61P 29/00; A61P 11/00; A61K 38/164; A61K 31/7088; A61K 31/4745; A61K 31/7125; A61K 31/7052; A61K 31/05; A61K 31/4178; A61K 38/1709

(57) ABSTRACT

Embodiments of the disclosure concern methods and compositions of treating or preventing viral infection, including of SARS-CoV-2, for example. In specific embodiments, one or more adjuvants are delivered to an individual receiving and/or having received fibroblasts and/or fibroblast-derived exosomes. In specific cases, the adjuvants comprise particular peptides, chloroquine and/or hydroxychloroquine, and/or one or more activators of one or more toll like receptors.

This application claims priority to U.S. Provisional Patent Application Ser. No. 63/002,134, filed Mar. 30, 2020, and claims priority to U.S. Provisional Patent Application Ser. No. 63/003,742, filed Apr. 1, 2020, both of which applications are incorporated by reference herein in their entirety.

### TECHNICAL FIELD

Embodiments of the disclosure include at least the fields of cell biology, molecular biology, physiology, and medicine.

### BACKGROUND

SARS-CoV-2 (previously known as 2019-nCoV), is a highly contagious coronavirus that is spreading rapidly around the world, causing a sharp rise of a pneumonia-like disease termed Coronavirus Disease 2019 (COVID-19) [1, 2]. COVID-19 presents a high mortality rate, estimated at 3.4% by the World Health Organization [3]. The rapid spread of the virus (estimated reproductive number R₀ 2.2-3.6 [4, 5] is causing a significant surge of patients requiring intensive care. More than 1 out of 4 hospitalized COVID-19 patients have required admission to an Intensive Care Unit (ICU) for respiratory support, and a large proportion of these ICU-COVID-19 patients, between 17% and 46%, have died [6-10].

A common observation among patients with severe COVID-19 infection is an inflammatory response localized to the lower respiratory tract [11-13]. This inflammation, associated with dyspnea and hypoxemia, in some cases evolves into excessive immune response with cytokine storm, determining progression to Acute Lung Injury (ALI), Acute Respiratory Distress Syndrome (ARDS), organ failure, and death [2, 10]. Draconian measures have been put in place in an attempt to curtail the impact of the COVID-19 epidemic on population health and healthcare systems. However, WHO has now classified COVID-19 a pandemic [3].

Crucially, no options are available for those patients with rapidly progressing ARDS evolving to organ failure. Although supportive care is provided whenever possible, including mechanical [14] ventilation and support of vital organ functions, it is insufficient in most severe cases. Therefore, there is an urgent need for novel therapies that can dampen the excessive inflammatory response in the lungs, associated with the immunopathological cytokine storm, and accelerate the regeneration of functional lung tissue in COVID-19 patients.

SARS-CoV-2, the virus initiating COVID-19, originated in the Wuhan province of China, believed possess a natural reservoir in bats, which may have been transferred to humans through a pangolin intermediary. This coronavirus, is a positive-sense, single-strand 30 kb RNA virus of the Coronaviridae family, it is approximately 50-200 nm in diameter. This virus exhibits some similarities to other coronaviruses such as SARS (2003) and MERS (2006). The SARS-Cov-2 RNA genome codes for at least 10 proteins including spike protein, orflab polyprotein, surface glycoprotein, ORF3a protein, envelope protein, membrane glycoprotein, ORF6 protein, ORF7a protein, ORF8 protein, nucleocapsid phosphoprotein, and ORF10 protein.

Affinity of SARS-Cov-2 to the human angiotensin converting enzyme 2 (ACE2) receptor and a polybasic cleavage site contributes to the transmission and pathogenicity of the virus. The primary pathology of COVID-19 is pulmonary because the virus has an affinity to the ACE2 receptor, which is found in high concentrations in the type II alveolar epithelial cells in the lungs. After internalization of the virus into these cells, viral replication occurs over several days during the replicative phase of this disease, during which only mild symptoms may be present. When the innate immune response fails to contain this virus, the adaptive immune response begins to effectively reduce viral titers by inducing a substantial release of inflammatory cytokines, signaling mediators that cause inflammation and increased activity of immune cells. This massive cytokine storm disrupts the epithelial-endothelial cell barrier between the alveoli and the pulmonary microvasculature causing diffuse alveolar damage and allowing proteinaceous fluid to accumulate in the alveoli. These inflammatory cytokines also damage the cells responsible for clearance of this fluid from the alveoli. The pattern of damage to the cells also seems to indicate a direct viral effect in addition to the hyperinflammatory injury. ALI can progress to ARDS as accumulation of fluid in the lungs obviously interferes with normal oxygenation of the blood causing hypoxemia and hypercapnia. The increased intrapulmonary pressure also increases the mean pulmonary arterial pressure causing acute cor pulmonale.

Adult Respiratory Distress Syndrome (ARDS) is a fulminant form of respiratory failure affecting many seriously ill patients. The early manifestations of ARDS are caused by increased permeability of the alveolo-capillary barrier leading to pulmonary edema, stiff lungs, and a large right-to-left intrapulmonary shunt. Polymorphonuclear leukocytes (PMNS) are involved in the pathogenesis of most ARDS, and multiple PMN mechanisms can effect pulmonary injury; interactions between PMN adherence, proteolytic enzyme release, and oxygen radical production are emphasized. ARDS therapy remains largely supportive and has had little impact on mortality [15].

The present disclosure provides solutions for the long-felt need in the art for viral infection and/or acute respiratory distress syndrome.

### BRIEF SUMMARY

The present disclosure is directed to systems, methods, and compositions for treatment of viral infection and/or ARDS. The viral infection may be of any kind, but in specific cases the virus is coronavirus, including at least the virus that causes MERS, SARS, and COVID-19. Particular embodiments of the disclosure include one or more adjuvants for a viral therapy, including, for example, a viral therapy comprising fibroblasts.

Embodiments of the disclosure include methods of treating or preventing a viral infection and/or acute respiratory distress syndrome (ARDS) in an individual, comprising administering to the individual a therapeutically effective amount of one or more adjuvants and a therapeutically effective amount of fibroblasts and/or fibroblast-derived products, including at least exosomes. The virus in specific cases may be adenovirus, alphavirus, BK virus, Bocavirus, calicivirus, coronavirus (including SARS-CoV or SARS-CoV-2), cytomegalovirus, distemper virus, Ebola virus, enterovirus, Epstein Barr virus, Varicella-zoster virus, flavivirus, hepatitis virus (AE), herpesvirus, infectious peritonitis virus, influenza virus, John Cunningham virus, leukemia virus, Lymphocytic choriomeningitis virus, Marburg virus, metapneumovirus, norovirus, orthomyxovirus, papilloma virus, parainfluenza virus, paramyxovirus, parvovirus, pestivirus, picorna virus, pox virus, rabies virus, reovirus, retrovirus, rhinovirus, Respiratory Syncytial Virus, rotavirus, West Nile virus, Zika virus, a virus that causes the common cold, or a virus that causes cancer.

In embodiments pursuant to ARDS, the ARDS may be caused by one or more factors selected from the group consisting of: a) cytokine storm; b) immunological cell infiltration; c) bacterial infection; d) viral infection; e) systemic inflammatory response syndrome; f) systemic inflammation; g) acute radiation syndrome; h) sepsis; and i) a combination thereof.

In some embodiments, there is a method of reprogramming monocytes in the lung of an individual suffering from or having a risk for ARDS, comprising administering to the individual an effective amount of one or more adjuvants with a therapeutically effective amount of fibroblasts and/or fibroblast-derived products, including at least exosomes, wherein said adjuvant comprises one or more peptides, one or more activators of one or more toll like receptors, chloroquine and/or hydroxychloroquine, resveratrol, losartan, azithromycin, or a mixture thereof.

In any method or composition herein, the adjuvants may comprise one or more peptides, including at least peptides selected from the group consisting of BPC-157; beta thymosine; Pam3CysSerLys4; functional derivatives thereof; and a combination thereof. The adjuvants may comprise one or more activators of one or more toll like receptors. For example, for toll like receptor 2, the activator of toll like receptor 2 may be selected from the group consisting of PAM2CSK4; beta glucan; water insoluble fractions of medicinal mushrooms (Lentinula edodes, Grifola frondosa, Hypsizygus marmoreus varieties, Flammulina velutipes); Diprovocim; HSPA4; HSPA5; HSPA9; HSPA13; HSPD1; VCAN; Lipoproteins LprG and LpqH; MTB lipoprotein Rv1016c; HKLM; FSL-1; and a combination thereof. For toll like receptor 3, an activator of toll like receptor 3 may be selected from the group consisting of Poly IC; ARNAX; double-stranded RNA; and a combination thereof. For TLR-4, the activator of TLR-4 may be LPS, Buprenorphine, Carbamazepine, Fentanyl, Levorphanol, Methadone, Cocaine, Morphine, Oxcarbazepine, Oxycodone, Pethidine, Glucuronoxylomannan from Cryptococcus, Morphine-3-glucuronide, lipoteichoic acid, β-defensin 2, small molecular weight hyaluronic acid, fibronectin EDA, snapin, tenascin C, or a combination thereof. For TLR-5, the activator of TLR-5 may be flagellin. For TLR-6, the activator may be FSL-1. For TLR-7, the activator of TLR-7 may be imiquimod. For TLR-8, the activator of TLR8 may be ssRNA40/LyoVec. For TLR-9, the activator of TLR-9 may be a CpG oligonucleotide, ODN2006, and/or Agatolimod.

In some embodiments, the adjuvant is chloroquine and/or hydroxychloroquine or functionally active derivatives thereof. The hydroxychloroquine may be administered under conditions (e.g., at a concentration and frequency) sufficient to reduce viral replication. The hydroxychloroquine may be administered under conditions sufficient to reduce activation of a TLR, such as TLR-9. The hydroxychloroquine may be administered under conditions sufficient to protect pulmonary type 2 epithelial cells. In specific embodiments, the hydroxychloroquine is administered under conditions sufficient to reduce production of one or more inflammatory cytokines in the lung. The inflammatory cytokine may be selected from the group consisting of interleukin-1; interleukin-6; interleukin-8; interleukin-11; interleukin-15; interleukin-17; interleukin-18; interleukin-23; TNF-alpha; angiopoietin; HMGB-1; and a combination thereof. In some cases, the adjuvant is resveratrol, losartan, and/or azithromycin or functionally active derivatives thereof. In specific cases, the functionally active derivative of chloroquine or hydroxychloroquine is SKM13, SKM14, a metal-chloroquine, or a combination thereof. In specific cases, functionally active derivative of resveratrol is trans-resveratrol (3,5,4′-trihydroxystilbene); cis-resveratrol; Pterostilbene (3,5-Dimethoxy-4′ Hydroxystilbene); Trimethoxystilbene; Tetramethoxystilbene; Pentamethoxystilbene; Dihydroxystilbene; Tetrahydroxystilbene; Hexahydroxystilbene; 4′-Bromoresveratrol; 3,4,5-Trimethoxy-4′-bromo-trans-stilbene (BTS); 3,4,5-Trimethoxy-4′-bromo-cis-stilbene (BCS); 2-Chlororesveratrol; 4-Iodoresveratrol; or a combination thereof. In specific cases, the functionally active derivative of losartan is a Losartan Nitroderivative. In specific cases, the functionally active derivative of azithromycin is 4″-O-(benzamido)alkyl carbamates of 11,12-cyclic carbonate AZM; 4″-O-(benzamido)butyl carbamates of 11,12-cyclic carbonate AZM; or combinations thereof.

With respect to the fibroblasts, they may be allogeneic, autologous or syngeneic with respect to the individual. In some cases, the fibroblasts are exposed to low level laser irradiation prior to administration. The fibroblasts may be derived from a source of tissues selected from the group consisting of a) dermal; b) placental; c) hair follicle; d) deciduous tooth; e) omentum; f) placenta; g) Wharton's jelly; h) bone marrow; i) adipose tissue; j) amniotic membrane; k) amniotic fluid; l) peripheral blood; and m) a combination thereof. The peripheral blood may be mobilized to enhance concentration of fibroblasts before isolation of fibroblasts. The mobilization may be achieved by treatment of the individual with one or more agents selected from the group consisting of a) G-CSF; b) M-CSF; c) GM-CSF; d) Mozibil; e) flt-3 ligand; and f) a combination thereof. In particular embodiments, the fibroblasts express CD73.

Any fibroblasts for any method encompassed herein may be administered intravenously, intranasally, intratracheally, or a combination thereof, as examples. Any fibroblasts may be pre-activated with one or more agents capable of enhancing fibroblast therapeutic activity, such as selected from the group consisting of a) mobility towards a chemotactic agent; b) production of anti-inflammatory agents; c) production of anti-apoptotic agents and d) a combination thereof. The mobility towards a chemotactic agent may be mediated by enhanced expression of a receptor associated with enhanced chemotaxis. The receptor associated with enhanced chemotaxis may be CXCR4. Anti-inflammatory factors may be selected from the group consisting of a) IL-4; b) IL-10; c) IL-13; d) IL-20; e) IL-27; f) IL-35; g) PGE-2; h) indolamine 2,3 deoxygenase; i) TGF-beta; j) EGF; and k) a combination thereof. In some cases, fibroblasts are modified to express an enhanced level of one or more therapeutic cytokines, such as selected from the group consisting of a) one or more cytokines that inhibit apoptosis; b) one or more cytokines that act as growth factors; c) one or more cytokines that act as immune modulators/anti-inflammatory agents; and d) a combination thereof. Cytokines that inhibit apoptosis may be selected from the group consisting of a) EGF; b) VEGF; c) angiopoietin; and d) a combination thereof. Cytokines that act as growth factors may be selected from the group consisting of a) HGF; b) FGF-1; c) FGF-2; d) KGF; e) CTNF; and f) a combination thereof. Cytokines that act as immune modulators/anti-inflammatory agents may be selected from the group consisting of a) IL-4; b) IL-10; c) IL-13; d) IL-20; e) IL-27; f) IL-35; g) PGE-2; h) indolamine 2,3 deoxygenase; i) TGF-beta; j) neuroaminidase; and k) a combination thereof. The fibroblasts may be endowed with ability to suppress viral infection, such as by production of interferon, including interferon selected from the group consisting of a) interferon alpha; b) interferon beta; c) interferon gamma; d) interferon tau; e) interferon omega; and f) a combination thereof. In cases wherein exosomes from fibroblasts are derived, the exosomes may express CD8 and/or annexin-V.

In certain cases, the individual is administered an additional therapy than the fibroblasts, fibroblast-derived products (including exosomes), and/or adjuvant(s). The additional therapy may be administered sequentially or simultaneously with the fibroblasts and/or one or more adjuvants. In some cases, the additional therapy is ventilation, a glucocorticoid, a surfactant, inhaled nitric oxide, an antioxidant, a protease inhibitor, a recombinant human activated protein C, a .beta.2-agonist, lisofylline, a statin, inhaled heparin, a diuretic, a sedative, an analgesic, a muscle relaxant, an antibiotic, inhaled prostacyclin, inhaled synthetic prostacydin analog, ketoconazole, alprostadil, keratinocyte growth factor, beta-agonists, human monoclonal antibody (mAb) against tissue factor VIIa (TS factor 7a), interferon receptor agonists, insulin, perfluorocarbon, budesonide, recombinant human angiotensin-converting enzyme (ACE), recombinant human Clara cell 10 kDa (CC10) protein, tissue plasminogen activator, human mesenchymal stem cells, nutritional therapy, methylprednisolone, dexamethasone, prednisone, prednisolone, betamethasone, triamcinolone, triamcinolone acetonide, beclometasone, albuterol, lisofylline, rosuvastatin, inhaled heparin, inhaled nitric oxide, recombinant human activated protein C, ibuprofen, naproxen, acetaminophen, cisatracurium besylate, procysteine, acetylcysteine, inhaled prostacydin, ketoconazole, alprostadil, keratinocyte growth factor, beta-agonists, human monoclonal antibody (mAb) against tissue factor VIIa (TS factor 7a), insulin, perfluorocarbon, budesonide, recombinant human angiotensin-converting enzyme (ACE), recombinant human Clara cell 10 kDa (CC10) protein, tissue plasminogen activator, human mesenchymal stem cells, a nutritional therapy, a combination of omega-3 fatty acids, antioxidants, gamma-linolenic acids with isocaloric foods, mechanical ventilation, or a combination thereof.

Embodiments encompass methods of treating or preventing infection with a viral infection and/or acute respiratory distress syndrome (ARDS; such as associated with the viral infection) comprising administering a therapeutically effective amount of a fibroblast cell population and administering one or more adjuvants. The population and the one or more adjuvants may or may not be in the same formulation and may or may not be administered to an individual in need thereof at the same time. The adjuvant(s) may be of any kind, but it one specific embodiment the adjuvant comprises particular peptides. In particular embodiments, the peptides are selected from the group consisting of a) BPC-157 (GEPPPGKPADDAGLV; SEQ ID NO:1); b) beta thymosine (SDKPDMAEI EKFDKSKLKK TETQEKNPLP SKETIEQEKQ AGES; SEQ ID NO:2); c) synthetic bacterial lipopeptide: Pam₃CysSerLys₄; d) functional derivatives thereof; and e) a combination thereof.

In particular embodiments, the adjuvants are activators of toll like receptors. In specific cases, the toll like receptor comprises toll like receptor 2, and in specific cases, the activator of toll like receptor 2 is selected from the group consisting of a) PAM2CSK4; b) beta glucan; c) water insoluble fractions of medicinal mushrooms (Lentinula edodes, Grifola frondosa, Hypsizygus marmoreus varieties, Flammulina velutipes); d) Diprovocim; e) HSPA4; f) HSPA5; g) HSPA9; h) HSPA13; i) HSPD1; j) VCAN; k) Lipoproteins LprG and LpqH; l) MTB lipoprotein Rv1016c; m) HKLM n) FSL-1; and o) a combination thereof. In some cases, the toll like receptor comprises toll like receptor 3, and in specific cases the activator of toll like receptor 3 is selected from the group consisting of a) Poly IC; b) ARNAX; c) double-stranded RNA; and d) a combination thereof.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIG. 1 shows suppression of pulmonary leakage by fibroblasts in an endotoxin ARDS model, as indicated by lung wet weight to body weight ratios (LWW/BW) that measures accumulation of lung water. From left to right, the bars represent control, lipopolysaccharides (LPS), bone marrow mesenchymal stem cells (BM-MSC), and fibroblasts.

FIG. 2 shows suppression of neutrophil infiltration by fibroblasts in an endotoxin ARDS model. From left to right, the bars represent control, LPS, BM-MSC, and fibroblasts.

FIG. 3 demonstrates suppression of TNF alpha by fibroblasts in an endotoxin ARDS model. From left to right, the bars represent control, LPS, BM-MSC, and fibroblasts.

FIG. 4 demonstrates suppression of IL-6 by fibroblasts in an endotoxin ARDS model. From left to right, the bars represent control, LPS, BM-MSC, and fibroblasts.

FIG. 5 shows that hydroxychloroquine anti-inflammatory efficacy increased by fibroblasts. From left to right, the bars represent control, macrophages, fibroblasts, and a combination of macrophages and fibroblasts.

FIG. 6 shows that hydroxychloroquine lung protection efficacy is increased by fibroblasts. From left to right, the bars represent control, hydroxychloroquine, fibroblasts, and a combination of hydroxychloroquine and fibroblasts.

### DETAILED DESCRIPTION

For the purposes of promoting an understanding of the principles of the novel technology, reference will now be made to the particular embodiments thereof, and specific language will be used to describe the same. It will nevertheless be understood that no limitation of the scope of the novel technology is thereby intended, such alterations, modifications, and further applications of the principles of the novel technology being contemplated as would normally occur to one skilled in the art to which the novel technology relates are within the scope of this disclosure and the claims.

### I. EXAMPLES OF DEFINITIONS

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value. In specific cases, unless explicitly stated otherwise or clearly implied otherwise the term ‘about’ refers to a range of values plus or minus 10 percent, e.g. about 1.0 encompasses values from 0.9 to 1.1.

As used herein, “allogeneic” refers to tissues or cells or other material from another body that in a natural setting are immunologically incompatible or capable of being immunologically incompatible, although from one or more individuals of the same species.

As used herein, “cell line” refers to a population of cells formed by one or more subcultivations of a primary cell culture. Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but not limited to seeding density, substrate, medium, growth conditions, and time between passaging.

As used herein, “conditioned medium” describes medium in which a specific cell or population of cells has been cultured for a period of time, and then removed, thus separating the medium from the cell or cells. When cells are cultured in a medium, they may secrete cellular factors that may be useful for any suitable purpose, such as may provide trophic support to other cells. Such trophic factors include, but are not limited to hormones, cytokines, chemokines, extracellular matrix (ECM), proteins, vesicles, antibodies, and granules. In this example, the medium containing the cellular factors is conditioned medium.

As used herein, a “trophic factor” describes a substance that promotes and/or supports survival, growth, proliferation and/or maturation of a cell. Alternatively or in addition, a trophic factor stimulates increased activity of a cell.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

As used herein, the terms “treatment,” “treat,” or “treating” refers to intervention in an attempt to alter the natural course of the individual or cell being treated, and may be performed either for prophylaxis or during the course of pathology of a disease or condition. Treatment may serve to accomplish one or more of various desired outcomes, including, for example, preventing occurrence or recurrence of disease, alleviation of symptoms, and diminishment of any direct or indirect pathological consequences of the disease, preventing metastasis, lowering the rate of disease progression, amelioration or palliation of the disease state, and remission or improved prognosis.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

A variety of aspects of this disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range as if explicitly written out. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. When ranges are present, the ranges may include the range endpoints.

The term “subject,” as used herein, may be used interchangeably with the term “individual” and generally refers to an individual in need of a therapy. The subject can be a mammal, such as a human, dog, cat, horse, pig or rodent. The subject can be a patient, e.g., have or be suspected of having or at risk for having a disease or medical condition related to bone. For subjects having or suspected of having a medical condition directly or indirectly associated with bone, the medical condition may be of one or more types. The subject may have a disease or be suspected of having the disease. The subject may be asymptomatic. The subject may be of any gender. The subject may be of a certain age, such as at least 30, 35, 40, 45, 50, 55, 60, 65, 70, 75, 80, 85, 90, 95, or 100 or more.

The term “fibroblast-derived product” (also “fibroblast-associated product”), as used herein, refers to a molecular or cellular agent derived or obtained from one or more fibroblasts. In some cases, a fibroblast-derived product is a molecular agent. Examples of molecular fibroblast-derived products include conditioned media from fibroblast culture, microvesicles obtained from fibroblasts, exosomes obtained from fibroblasts, apoptotic vesicles obtained from fibroblasts, nucleic acids (e.g., DNA, RNA, mRNA, miRNA, etc.) obtained from fibroblasts, proteins (e.g., growth factors, cytokines, etc.) obtained from fibroblasts, and lipids obtained from fibroblasts. In some cases, a fibroblast-derived product is a cellular agent. Examples of cellular fibroblast-derived products include cells (e.g., stem cells, hematopoietic cells, neural cells, etc.) produced by differentiation and/or de-differentiation of fibroblasts.

As used herein, the term “therapeutically effective amount” is synonymous with “effective amount”, “therapeutically effective dose”, and/or “effective dose” and refers to the amount of compound that will elicit the biological, cosmetic or clinical response being sought by the practitioner in an individual in need thereof. As one example, an effective amount is the amount sufficient to reduce immunogenicity of a group of cells. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly.

### II. EMBODIMENTS

Particular embodiments of the present disclosure concern augmentation of fibroblast therapy for viruses of any kind, including any coronavirus. Specific embodiments of the present disclosure concern peptides and adjuvants for augmentation of fibroblast therapy for viruses of any kind, including any coronavirus. Disclosed are means of augmenting therapeutic efficacy of fibroblasts in an individual who has a viral infection or is at risk for having a viral infection (including coronavirus infection) by administering one or more adjuvants, including at least certain peptides and other molecules. The peptides include stimulators of innate immunity, stimulators of adaptive immunity, as well as peptides involved in the modulation of the viral life cycle, and one or more may be provided to an individual having a viral infection, whether they are symptomatic or not, or at risk for a viral infection, such as having been exposed to an individual or environment or surface with the infection. In one embodiment, the administration of beta thymosine is utilized to enhance natural killer (NK) cell activity, while administration of interferon-producing fibroblasts is also or alternatively administered, including at least concurrently administered. In one embodiment, the disclosure provides means of preventing infection, propagation, and pathology caused by the COVID-19 virus.

Embodiments of the present disclosure may be utilized for any type of viral infection. In specific embodiments, the virus is adenovirus, alphavirus, BK virus, Bocavirus, calicivirus, coronavirus, cytomegalovirus, distemper virus, Ebola virus, enterovirus, Epstein Barr virus, Varicella-zoster virus, flavivirus, hepatitis virus (AE), herpesvirus, infectious peritonitis virus, influenza virus, John Cunningham virus, leukemia virus, Lymphocytic choriomeningitis virus, Marburg virus, metapneumovirus, norovirus, orthomyxovirus, papilloma virus, parainfluenza virus, paramyxovirus, parvovirus, pestivirus, picorna virus, pox virus, rabies virus, reovirus, retrovirus, rhinovirus, Respiratory Syncytial Virus, rotavirus, West Nile virus, Zika virus, a virus that causes the common cold, or a virus that causes cancer. The virus may be a DNA virus, RNA virus, or reverse transcribing virus. They may be single stranded or double stranded. Viruses from any viral infection may be treated, including from one of the following families: Adenoviridae, Picornaviridae, Herpesviridae, Hepadnaviridae, Flaviviridae, Retroviridae, Orthomyxoviridae, Paramyxoviridae, Papovaviridae, Polyomavirus, Rhabdoviridae, or Togaviridae. In a specific embodiment, the virus is a coronavirus and may be of any kind, including alpha, beta, gamma, and delta coronaviruses. Specific coronaviruses include at least SARS-CoV-2 (the novel coronavirus that causes coronavirus disease 2019, or COVID-19); SARS-CoV (the beta coronavirus that causes severe acute respiratory syndrome, or SARS); MERS-CoV (the beta coronavirus that causes Middle East Respiratory Syndrome, or MERS); 229E (alpha coronavirus); NL63 (alpha coronavirus); OC43 (beta coronavirus); and HKU1 (beta coronavirus).

In particular embodiments, fibroblast therapy for an individual may be administered to the individual prior to, at the same time as, and/or following administration of one or more adjuvants to the individual.

In one embodiment, the disclosure provides immune modulatory peptides as compositions useful to augment the therapeutic effects of fibroblasts in viral infections. In one embodiment, the disclosure provides the use of BPC-157 together with fibroblasts, such as to induce acceleration of pulmonary healing subsequent to viral infection, and/or development of ARDS. It is known that ARDS is associated with numerous inflammatory cytokines, such as TNF-alpha [16]. The causative role of TNF-alpha in ARDS can be seen in studies in which administration of this cytokine leads to activation of neutrophils and a pathology that resembles ARDS [17]. TNF-alpha has been found elevated in patients with ARDS. The inflammatory cytokine IL-1 beta has also been associated with ARDS [18-23]. TNF-alpha and IL-1 are both increased during hypoxia, and hypoxia occurs because of ARDS [24] This was shown in a study in which exposure to hypoxia (PO2=9+/−1 torr) increased human peripheral blood mononuclear cell production and secretion of interleukin-1 (IL-1)alpha, IL-1 beta, and tumor necrosis factor (TNF) percent of control=190% for IL-1 alpha, p=0.014; 219% for IL-1 beta, p=0.014; and 243% for TNF, p=0.037) following treatment with endotoxin (1 ng/ml). Hypoxia potentiated the increased production of these inflammatory cytokines at subthreshold levels of endotoxin with potentiation increasing at lower O2 concentrations. Hypoxia also increased cytokine production induced by the tumor promoter phorbol myristate acetate, suggesting a generalized biologic response. It was concluded that hypoxia increases IL-1 and TNF production and speculate that this mechanism aggravates a variety of pathologic conditions involving endotoxin such as adult respiratory distress syndrome (ARDS), multiple organ failure, and septic shock [25]. In specific embodiments, administration of the therapy and adjuvants encompassed herein results in a reduction in the level of TNF-alpha and/or IL-1, at least.

In some embodiments, one or more peptides are utilized as adjuvants in conjunction with fibroblast therapy. In some cases, the peptide comprises BPC-157 (GEPPPGKPADDAGLV; SEQ ID NO:1) or a functional derivative thereof. The peptide may comprise, consist of, or consist essentially of SEQ ID NO:1. In some cases, the peptide comprises SEQ ID NO:1 but also comprises an N-terminal extension and/or a C-terminal extension. The peptide comprising SEQ ID NO:1 may be 16, 17, 18, 19, 20, 25, 30, 35, 40, 45, 50, or more amino acids in length. In some cases the peptide comprises a sequence having 1, 2, 3, 4, or 5 or more amino acid substitutions compared to SEQ ID NO:1, and the substitutions may or may not be conservative. A functional derivative of SEQ ID NO:1 may be at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to SEQ ID NO:1.

In some cases, the peptide comprises beta thymosine (SDKPDMAEI EKFDKSKLKK TETQEKNPLP SKETIEQEKQ AGES; SEQ ID NO:2) or a functional derivative thereof. The peptide may comprise, consist of, or consist essentially of SEQ ID NO:2. In some cases, the peptide comprises SEQ ID NO:2 but also comprises an N-terminal extension and/or a C-terminal extension. The peptide comprising SEQ ID NO:1 may be 44, 45, 46, 47, 48, 49, 50, 51, 52, 53, 54, 55, 56, 57, 58, 59, 60, 61, 62, 63, 64, 65, or more amino acids in length. In some cases the peptide comprises a sequence having 1, 2, 3, 4, 5, 6, 7, 8, 9, 10, or more amino acid substitutions compared to SEQ ID NO:2, and the substitutions may or may not be conservative. A functional derivative of SEQ ID NO:2 may be at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to SEQ ID NO:2.

In some cases, the adjuvant comprises the synthetic bacterial lipopeptide: Pam₃CysSerLys₄ (Abcam®) or a functional derivative thereof. In some cases, the peptide portion of the molecule (CysSerLys₄) comprises 1, 2, or more substitutions, whether conservative or not. The peptide may also be extended in length an additional 1, 2, 3, 4, 5, 6, 7, 8, 9, 10, or more amino acids, for example.

In some embodiments, the disclosure provides the adjuvant use of other substances together with fibroblasts for treatment of any virus, including at least coronaviruses. The substances include prostacyclin analogues such as iloprost and cicaprost [26]. Other substances encompassed for augmentation of fibroblast anti-coronaviral activity includes agents that modify macrophage activity, and/or stimulate innate immune system activity [27], and said agents include RRx-001 [28], the bee venom derived peptide melittin [29], CpG DNA [30, 31], metformin [32], Chinese medicine derivative puerarin [33], rhubarb derivative emodin [34], dietary supplement chlorogenic acid [35], propranolol [36], poly ICLC [37], BCG [38], Agaricus blazei Murill mushroom extract [39], endotoxin [40], olive skin derivative maslinic acid [41], intravenous immunoglobulin [42], phosphotidylserine targeting antibodies [43], dimethyl sulfoxide [44], surfactant protein A [45], Zoledronic acid [46], bacteriophages [47], beta glucan and extracts of medicinal mushrooms [48], Diprovocim [49], HSPA4, HSPA5, HSPA9, HSPA13, HSPD1, and VCAN [50], Lipoproteins LprG and LpqH [51], MTB lipoprotein Rv1016c [52], FSL-1 [53], pentraxin 3 [54], and/or ARNAX [55].

In some embodiments, fibroblasts, together with therapeutic adjuvant(s), are administered to prevent ARDS, which is a life-threatening lung injury that allows fluid to leak into the lungs. Breathing becomes difficult and oxygen cannot get into the body. Most people who get ARDS are already at the hospital for trauma or illness. The lung injury causing ARDS is characterized by injury to the lung epithelium that leads to impaired resolution of pulmonary edema and also facilitates accumulation of protein-rich edema fluid and inflammatory cells in the distal airspaces of the lung Inflammatory mediators produced by neutrophils and macrophages as well as viruses, cause damage to the tight junctions between alveolar epithelial cells allowing pathologic flow of proteinaceous fluid into alveoli. Normal alveolar fluid clearance from the alveoli to the interstitium adequately removes any fluid accumulation, but the rate of fluid clearance is impaired by infection, inflammatory cytokines and the mechanical ventilation frequently employed in ARDS. Cytokine storm decreases the number of α-epithelial sodium channel (α-ENaC) subunits in the apical membrane of alveolar epithelial cells, which contributes to increased accumulation and impaired clearance of fluid from the alveoli. Exposure of cultured type II alveolar epithelial cells to IL-1(3, TNF-α, and IFN-γ increases the protein permeability of alveoli by 5-fold over 24 hours. Impaired pulmonary function due to pulmonary capillary endothelial and alveolar epithelial cell dysfunction is exacerbated by damage to type II alveolar cells (pulmonary progenitor cells), which also interferes with normal surfactant function. Damage to the delicate alveolar-capillary barrier causes fluid accumulation in the air spaces of the lungs, significantly interfering with gas exchange in the alveoli and the clearance of the fluid. Normally, pulmonary capillary endothelial cells form a tight barrier that separates the pulmonary capillaries and the alveoli, which prevents the passage of proteinaceous fluid and inflammatory cells between these cells. Adherens junctions, formed by the association of VE-cadherin proteins on the membrane of adjacent endothelial cells, create the alveolar-capillary barrier Inflammatory cytokines and other signaling proteins present in cytokine storm disrupt these adherens junctions between endothelial cells allowing leakage of the capillaries. Neutrophils, activated platelets and bacterial products, such as endotoxin, can also damage or destroy the endothelial cells themselves, further increasing the permeability of this barrier. In some embodiments of the disclosure, the combination of fibroblasts and adjuvants is used to decrease inflammation in the lung, so methods of decreasing lung inflammation are encompassed herein. In other embodiments, the combination of fibroblasts and adjuvants is used to induce an increase in surfactant production, so methods of increasing surfactant production are encompassed herein. In other embodiments, the disclosure provides means of suppressing the loss of alveoli through protecting the type 2 pulmonary epithelial cells that are the target of coronaviruses, so methods of suppressing the loss of alveoli, such as through protecting type 2 pulmonary epithelial cells that are the target of coronavirus, are encompassed herein.

ARDS is characterized by diffuse alveolar damage that causes interstitial and alveolar edema, which progresses through exudative, proliferative, and fibrotic phases to pulmonary fibrosis. Accumulation and impaired clearance of proteinaceous fluid in the alveoli, and the cardiac sequelae result in acute onset of tachypnea, hypoxemia, and loss of compliance of the lungs. Severity of ARDS can be characterized using various measures, such as AECC and Berlin criteria or Lung Injury Scores, based on PaO₂/FiO₂ and other clinical data. Biomarkers such as soluble intercellular adhesion molecule-1, von Willebrand factor antigen, IL-6, IL-8, SP-D, PAI-1 and TNFR1 can provide additional information about the progression of the disease. In almost 50% of cases, hypoxemia can progress to become more severe, despite increasing ventilatory support and supplemental oxygen, eventually leading to complete respiratory failure. The cytokine storm that precipitates ARDS also affects the pulmonary circulation causing diffuse pulmonary endothelial injury and thrombus formation. Fibrosis of the pulmonary endothelium can occlude these alveolar capillaries and other small vessels and contribute to pulmonary hypertension. Hypoxemia and hypercapnia, which alter vasomotor tone, increased intrathoracic pressure due to ventilator support and thrombosis all exacerbate this pulmonary hypertension leading to secondary cardiac effects such as cor pulmonale.

The role of inflammatory cytokines in the progression of ARDS and its pathology may be seen in several situations. For example, tumor necrosis factor (TNF)-alpha, has been demonstrated to correlate with severity of ARDS in several studies. In one study, measure of plasma TNF alpha levels (pl-TNF alpha) in 34 patients with ARDS and in 16 controls was examined. Plasma, TNF alpha was elevated in 76% of the patients with ARDS (71+/−10⁴ pg/ml) and in 48% of the at-risk patients (47+/−73 pg/ml), providing some indication that TNF-alpha may correlated with ARDS [56]. In another study assessment of TNF-alpha was performed in fourteen hospitalized patients with a diagnosis of SARS-associated coronavirus infection. All patients had fever, dry cough and dyspnea. Twelve were intubated during hospitalization. The median duration from onset of fever to the nadir level or most severe condition was 9 days for hypoxia. The 8 patients who died possessed significantly higher peak levels of serum TNF-alpha compared to those who survived (14 vs 9.1 pg/mL; p=0.06) [57]. Another study demonstrated correlation between TNF-alpha and mortality. The study examined ICU patients on ventilator with (n=9) and without (n=12) evidence of ARDS. The median peak TNF concentration in control patients was 40 ng/L (range less than 40-100 ng/L) and in ARDS patients 231 ng/L (range 100-2550 ng/L; p less than 0.001). All of the control patients were discharged alive from the ICU, whereas 6 of 9 ARDS patients died in the ICU. In 6 ARDS patients, it was possible to measure more than 4 consecutive plasma TNF levels. Of these 6 patients, the 3 with persistent elevations in systemic TNF above 230 ng/L succumbed (p less than 0.05, one-tailed) [58].

It is believed the TNF-alpha production causes pathology in ARDS at several levels. In one experiment, TNF-alpha was administered intra-tracheally at 500 ng in healthy rats. It was observed that within 5 hours, lung lavage neutrophils, lung myeloperoxidase (MPO) activity, and lung leak was substantially higher in the treated as compared to saline-treated control rats [59]. In another study, it was shown that TNF-alpha maintains viability of neutrophils, thus allowing them to produce exaggerated inflammation responses. Scientists exposed neutrophils TNFalpha (100 ng/mL) in the presence or absence of antibodies to IL-8, and the extent of apoptosis was assessed. An enzyme-linked immunoassay was used to measure levels of the anti-apoptotic cytokine IL-8, induced by TNFalpha-stimulation. Because TNFalpha may mediate its effect through various cell-signaling pathways, the study next assessed the effect of kinase inhibition on the ability of TNFalpha to effect apoptosis and IL-8 production. Treatment with TNFalpha had a biphasic effect: at 4-8 h, apoptosis was increased but was markedly suppressed at 24 h (P<0.05). PMN cultured for 24 h with TNFalpha also showed markedly increased levels of IL-8. Neutralization of IL-8 inhibited the ability of TNFalpha to suppress apoptosis (P<0.05). These data illustrate a novel mechanism by which TNFalpha can indirectly elicit an anti-apoptotic effect via release of the anti-apoptotic chemokine IL-8 [60]. One intriguing evidence that TNF-alpha is a potential cause of ARDS are studies in which TNF-alpha was administered systemically as a cancer therapeutic, and one of the adverse effects observed in some patients was a ARDS-type pathology [61].

Another cytokine that has been studied extensively in ARDS is interleukin-6. This cytokine is known to possess pro-inflammatory properties [62], as well as to suppress generation of T regulatory cells and promote Th17 cells [63-65]. It is accepted that in ARDS there is a reduction in T regulatory cells [66], whose role is tissue protection [67], and Th17 cells, which are commonly associated with inflammation [68]. In one study, 27 consecutive patients with severe medical ARDS. Plasma levels of tumor necrosis factor alpha (TNF-alpha) and interleukins (ILs) 1 beta, 2, 4, 6, and 8 were measured (enzyme-linked immunosorbent assay [ELISA] method) on days 1, 2, 3, 5, 7, 10, and 12 of ARDS and every third day thereafter while patients were receiving mechanical ventilation. Subgroups of patients were identified based on outcome, cause of ARDS, presence or absence of sepsis, shock, and MODS at the time ARDS developed. Subgroups were compared for levels of plasma inflammatory cytokines on day 1 of ARDS and over time. Of the 27 patients, 13 survived ICU admission and 14 died (a mortality rate of 52%). Overall mortality was higher in patients with sepsis (86 vs 38%, p<0.02). The mean initial plasma levels of TNF-alpha, IL-1 beta, IL-6, and IL-8 were significantly higher in nonsurvivors (p<0.0001) and in those patients with sepsis (p<0.0001). Plasma levels of IL-1 beta (p<0.01) and IL-6 (p=0.03) were more strongly associated with patient outcome than cause of ARDS (p=0.8), lung injury score (LIS), APACHE II score, sepsis (p=0.16), shock, or MODS score. Plasma levels of TNF-alpha, IL-1 beta, IL-6, and IL-8 remained significantly elevated over time (p<0.0001) in those who died. This study strongly supports the addition of IL-6 as another cytokine mediatory involved in the pathogenesis of ARDS [69].

A subsequent study examined 24 ARDS patients with MODS (ARDS+MODS group), 18 patients with ARDS but without MODS (ARDS group), and 55 patients with MODS but without ARDS as controls (control group). It was found that serum IL-6 levels in the ARDS+MODS group were significantly higher than those in the ARDS and MODS groups (P<0.01). The IL-6 levels increased with elevated ARDS illness severity (P<0.01); the sensitivity of IL-6 was high in all groups. Moreover, the IL-6 values were closely associated with patient survival [70]. Several other studies have shown correlation between IL-6 elevation and poor prognosis in ARDS [71-73].

In one embodiment, compositions of the present disclosure may be obtained from isolated fibroblast cells or a population thereof capable of proliferating and differentiating into ectoderm, mesoderm, or endoderm. In some embodiments, an isolated fibroblast cell expresses at least one of Oct-4, Nanog, Sox-2, KLF4, c-Myc, Rex-1, GDF-3, LIF receptor, CD105, CD117, CD344 or Stella markers. In some embodiments, an isolated fibroblast cell does not express at least one of MHC class I, MHC class II, CD45, CD13, CD49c, CD66b, CD73 (although in alternative cases the fibroblast is CD73+), CD105, or CD90 cell surface proteins. Such isolated fibroblast cells may be used as a source of products of any kind, including at least conditioned media. The cells may be cultured alone, or may by cultured in the presence of other cells in order to further upregulate production of growth factors in the conditioned media. Fibroblasts may be expanded and utilized by administration themselves, or may be cultured in a growth media in order to obtain conditioned media. The term Growth Medium generally refers to a medium sufficient for the culturing of fibroblasts. In particular, one particular medium for the culturing of the cells of the disclosure herein comprises Dulbecco's Modified Essential Media (DMEM). Specific media may be DMEM-low glucose (also DMEM-LG herein) (Invitrogen®, Carlsbad, Calif.). The DMEM-low glucose may be supplemented with 15% (v/v) fetal bovine serum (e.g., defined fetal bovine serum, Hyclone™, Logan Utah), antibiotics/antimycotics (penicillin (100 Units/milliliter), streptomycin (100 milligrams/milliliter), and amphotericin B (0.25 micrograms/milliliter), (Invitrogen®, Carlsbad, Calif.)), and 0.001% (v/v) 2-mercaptoethanol (Sigma®, St. Louis Mo.). In some cases, different growth media are used, or different supplementations are provided, and these are normally indicated as supplementations to Growth Medium. Also relating to the present invention, the term standard growth conditions, as used herein refers to culturing of cells at 37° C., in a standard atmosphere comprising 5% CO₂, where relative humidity is maintained at about 100%. While the foregoing conditions are useful for culturing, it is to be understood that such conditions are capable of being varied by the skilled artisan who will appreciate the options available in the art for culturing cells, for example, varying the temperature, CO₂, relative humidity, oxygen, growth medium, and the like.

Also disclosed herein are cultured cells. Various terms are used to describe cells in culture. Cell culture refers generally to cells taken from a living organism and grown under controlled condition (“in culture” or “cultured”). A primary cell culture is a culture of cells, tissues, or organs taken directly from an organism(s) before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number, or the “doubling time”.

Fibroblast cells used in the disclosed methods can undergo at least 25, 30, 35, or 40 doublings prior to reaching a senescent state. Methods for deriving cells capable of doubling to reach 10¹⁴ cells or more are provided. Examples are those methods which derive cells that can double sufficiently to produce at least about 10¹⁴, 10¹⁵, 10¹⁶, or 10¹⁷ or more cells when seeded at from about 10³ to about 10⁶ cells/cm² in culture. Preferably these cell numbers are produced within 80, 70, or 60 days or less. In one embodiment, fibroblast cells used are isolated and expanded, and possess one or more markers selected from a group consisting of CD10, CD13, CD44, CD73, CD90, CD141, PDGFr-alpha, HLA-A, HLA-B, and HLA-C. In some embodiments, the fibroblast cells do not produce one or more of CD31, CD34, CD45, CD117, CD141, HLA-DR, HLA-DP, or HLA-DQ.

When referring to cultured cells, including fibroblast cells and vertebrae cells, the term senescence (also “replicative senescence” or “cellular senescence”) refers to a property attributable to finite cell cultures; namely, their inability to grow beyond a finite number of population doublings (sometimes referred to as Hayflick's limit). Although cellular senescence was first described using fibroblast-like cells, most normal human cell types that can be grown successfully in culture undergo cellular senescence. The in vitro lifespan of different cell types varies, but the maximum lifespan is typically fewer than 100 population doublings (this is the number of doublings for all the cells in the culture to become senescent and thus render the culture unable to divide). Senescence does not depend on chronological time, but rather is measured by the number of cell divisions, or population doublings, the culture has undergone. Thus, cells made quiescent by removing essential growth factors are able to resume growth and division when the growth factors are re-introduced, and thereafter carry out the same number of doublings as equivalent cells grown continuously. Similarly, when cells are frozen in liquid nitrogen after various numbers of population doublings and then thawed and cultured, they undergo substantially the same number of doublings as cells maintained unfrozen in culture. Senescent cells are not dead or dying cells; they are resistant to programmed cell death (apoptosis) and can be maintained in their nondividing state for as long as three years. These cells are alive and metabolically active, but they do not divide.

In some cases, fibroblast cells are obtained from a biopsy, and the donor providing the biopsy may be either the individual to be treated (autologous), or the donor may be different from the individual to be treated (allogeneic). In cases wherein allogeneic fibroblast cells are utilized for an individual, the fibroblast cells may come from one or a plurality of donors.

The fibroblasts may be obtained from a source selected from the group consisting of dermal fibroblasts; placental fibroblasts; adipose fibroblasts; bone marrow fibroblasts; foreskin fibroblasts; umbilical cord fibroblasts; hair follicle-derived fibroblasts; nail-derived fibroblasts; endometrial-derived fibroblasts; keloid-derived fibroblasts; and a combination thereof. In some embodiments, fibroblasts are dermal fibroblasts.

In some embodiments, fibroblasts are manipulated or stimulated to produce one or more factors. In some embodiments, fibroblasts are manipulated or stimulated to produce leukemia inhibitory factor (LIF), brain-derived neurotrophic factor (BDNF), epidermal growth factor receptor (EGF), basic fibroblast growth factor (bFGF), FGF-6, glial-derived neurotrophic factor (GDNF), granulocyte colony-stimulating factor (GCSF), hepatocyte growth factor (HGF), IFN-γ, insulin-like growth factor binding protein (IGFBP-2), IGFBP-6, IL-1ra, IL-6, IL-8, monocyte chemotactic protein (MCP-1), mononuclear phagocyte colony-stimulating factor (M-CSF), neurotrophic factors (NT3), tissue inhibitor of metalloproteinases (TIMP-1), TIMP-2, tumor necrosis factor (TNF-β), vascular endothelial growth factor (VEGF), VEGF-D, urokinase plasminogen activator receptor (uPAR), bone morphogenetic protein 4 (BMP4), IL1-a, IL-3, leptin, stem cell factor (SCF), stromal cell-derived factor-1 (SDF-1), platelet derived growth factor-BB (PDGFBB), transforming growth factors beta (TGFβ-1) and/or TGFβ-3. Factors from manipulated or stimulated fibroblasts may be present in conditioned media and collected for therapeutic use.

In some embodiments, fibroblasts are transfected with one or more angiogenic genes, such as to enhance ability to promote neural repair. An “angiogenic gene” describes a gene encoding for a protein or polypeptide capable of stimulating or enhancing angiogenesis in a culture system, tissue, or organism. Examples of angiogenic genes which may be useful in transfection of fibroblasts include activin A, adrenomedullin, aFGF, ALK1, ALK5, ANF, angiogenin, angiopoietin-1, angiopoietin-2, angiopoietin-3, angiopoietin-4, bFGF, B61, bFGF inducing activity, cadherins, CAM-RF, cGMP analogs, ChDI, CLAF, claudins, collagen, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shingoingolipid G-protein coupled receptor-1 (EDG1), ephrins, Epo, HGF, TGF-beta, PD-ECGF, PDGF, IGF, IL8, growth hormone, fibrin fragment E, FGF-5, fibronectin, fibronectin receptor, Factor X, HB-EGF, HBNF, HGF, HUAF, heart derived inhibitor of vascular cell proliferation, IL1, IGF-2 IFN-gamma, α1β1 integrin, α2β1 integrin, K-FGF, LIF, leiomyoma-derived growth factor, MCP-1, macrophage-derived growth factor, monocyte-derived growth factor, MD-ECI, MECIF, MMP2, MMP3, MMP9, urokiase plasminogen activator, neuropilin, neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (SIP1), Syk, SLP76, tachykinins, TGF-beta, Tie 1, Tie2, TGF-β, TGF-β receptors, TIMPs, TNF-α, transferrin, thrombospondin, urokinase, VEGF-A, VEGF-B, VEGF-C, VEGF-D, VEGF-E, VEGF, VEGF(164), VEGI, and EG-VEGF. Fibroblasts transfected with one or more angiogenic factors may be used in the disclosed methods of treatment or prevention of ARDS.

Under appropriate conditions, fibroblasts may be capable of producing interleukin-1 (IL-1) and/or other inflammatory cytokines. In some embodiments, fibroblasts of the present disclosure are modified (e.g., by gene editing) to prevent or reduce expression of IL-1 or other inflammatory cytokines. For example, in some embodiments, fibroblasts are fibroblasts having a deleted or non-functional IL-1 gene (for example, using CRISPR), such that the fibroblasts are unable to express IL-1. Such modified fibroblasts may be useful in the therapeutic methods of the present disclosure by having limited pro-inflammatory capabilities when provided to a subject. In some embodiments, fibroblasts are treated with (e.g., cultured with) TNF-α, thereby inducing expression of growth factors and/or fibroblast proliferation.

In some embodiments, fibroblasts of the present disclosure are used as precursor cells that differentiate following introduction into an individual (e.g., into the pulmonary cells of an individual). In some embodiments, fibroblasts are subjected to differentiation into a different cell type prior to introduction into the individual (e.g., into the lung system).

As disclosed herein, fibroblasts may secrete one or more factors prior to or following introduction into an individual. Such factors include, but are not limited to, growth factors, trophic factors and/or cytokines. In some instances, the secreted factors can have a therapeutic effect in the individual. In some embodiments, a secreted factor activates a particular cell. In some embodiments, the secreted factor activates neighboring and/or distal endogenous cells. In some embodiments, the secreted factor stimulated cell proliferation and/or cell differentiation. In some embodiments, fibroblasts secrete a cytokine or growth factor selected from human growth factor, fibroblast growth factor, nerve growth factor, insulin-like growth factors, hemopoietic stem cell growth factors, a member of the fibroblast growth factor family, a member of the platelet-derived growth factor family, a vascular or endothelial cell growth factor, and/or a member of the TGFβ family.

The present inventors have unexpectedly discovered that the administration route of the fibroblasts may be important to achieve therapeutic efficacy, in some embodiments. For instance, in the treatment of ARDS, a pharmaceutical composition comprising fibroblasts and/or extracellular vesicles derived from fibroblasts, and/or apoptotic bodies of the fibroblasts may be administered via peripheral intravenous injection, central venous injection into the right atrium, injection into the right ventricle of the heart, and/or injection into the pulmonary trunk/artery. Specifically, the pharmaceutical compositions as per the present disclosure display considerable therapeutic efficacy in the patient group that (1) suffers from ARDS, infant respiratory distress syndrome (IRDS), pulmonary hypertension (PH), or any other pulmonary disease or disorder within the scope of the present invention, and (2) are eligible and/or are undergoing extra-corporal membranous oxygenation (ECMO) treatment. The pharmaceutical compositions in accordance with the present disclosure can thus be administered both to patients that have already been placed on ECMO support, and to patients that are eligible but have not yet commenced ECMO treatment.

The present disclosure, in a further aspect, thus relates to the use of the pharmaceutical compositions according to the present disclosure for use in medicine, and specifically in the treatment of diseases and disorders such as ARDS, IRDS, PH, congenital heart diseases, and acute organ failure (optionally in connection with ARDS and/or IRDS), for instance heart failure, kidney failure, and/or liver failure.

ARDS is an important cause of acute respiratory failure and is often associated with multiple organ failure. Several clinical disorders can precipitate ARDS, for instance viral and/or bacterial pneumonia, aspiration of gastric contents, sepsis, surgery, and major trauma. The clinical criteria for ARDS are normally the following: Acute onset: 20-50% of acute lung injury patients will develop ARDS within 7 days. Capillary wedge pressure (PCWP) 518 mmHg or no evidence of cardiac failure. Chest X-ray shows bilateral infiltrates. Refractory hypoxaemia: ARDS is present when PaO.sub.2:FiO.sub.2<200. Physiologically, ARDS is characterized by increased permeability pulmonary edema, severe arterial hypoxemia, and impaired carbon dioxide excretion and is the result of an on-going inflammatory response. [0105] Clinically, up-regulation of inflammatory cytokines frequently persists in the patients.

IRDS of the newborn is the most common cause of respiratory distress in premature infants, correlating with structural and functional lung immaturity. The pathophysiology is characterized by an ongoing inflammatory response giving immature type II alveolar cells that produce less surfactant, causing an increase in alveolar surface tension and a decrease in compliance. The resultant atelectasis causes pulmonary vascular constriction, hypoperfusion, and lung tissue ischemia. Hyaline membranes form through the combination of sloughed epithelium, protein, and edema. Persistent respiratory distress syndrome leads to bronchopulmonary dysplasia, characterized by typical chest radiography findings and chronic oxygen dependence.

PH is an increase in blood pressure in the pulmonary artery, pulmonary vein, and/or the pulmonary capillaries and it can be a severe disease with a markedly decreased exercise tolerance and heart failure. Evidence is accumulating to suggest that inflammation plays a significant role in the pathogenesis of PH. Endothelial cells play an important role in inflammation and immune reactions, and inflammatory cytokines cause endothelial dysfunction. Endothelial dysfunction is a hallmark of PH, consisting in reduced availability of vasodilators and antiproliferative factors and increased production of vasoconstrictors and vascular proliferative factors. Up-regulation of inflammatory cytokines and perivascular inflammatory cell infiltration have been detected in the lungs of patients with PH. Persistent PH of the newborn occurs when pulmonary vascular resistance fails to decrease soon after birth as with normal transition. The etiology may be idiopathic or secondary to meconium aspiration syndrome, pneumonia or sepsis, respiratory distress syndrome, or transient tachypnea of the newborn. The increased pulmonary hypertension gives rise to an ongoing inflammatory response in the lung.

In one embodiment of the disclosure, stimulation of fibroblasts is performed by treatment with a toll like receptor prior to administration of fibroblasts. Numerous toll like receptors may be activated for use in the current invention. In a preferred embodiment, toll like receptors which recognize RNA, similar to ones which are activated by viruses are utilized. In a preferred embodiment, toll-like receptor 3 activators are administered to fibroblasts. Specific toll like receptors including Poly-IC.

In some embodiments, the methods concern treatment or prevention of infection with SARS-CoV-2 (also known as 2019-nCoV or COVID-19). The outbreak of novel coronavirus (2019-nCoV)—now known as Coronavirus Disease (COVID-19), has been declared by the WHO as a global health emergency. Initial infection occurs in the respiratory tract, in which the virus incubates until inducing carrier status, and/or pathological infection in the host. COVID-19 mortality is associated with acute respiratory distress syndrome (ARDS) and multiple organ failure [7, 74]. What is needed are treatments that can inhibit the infection at the site of entry and suppress viral propagation so as to prevent progression to advanced state.

Because it is known that patients with stronger lung-resident immune responses are more resilient to COVID-19 as compared to ones with weaker immunity, means of boosting localized immunity are urgently needed. The methods and compositions of the present disclosure are useful for those positive for COVID-19 or who have been exposed to an individual positive for COVID-19.

When cells are infected with viruses, they produce a family of chemicals called “interferons”, which “interfere” with ability of the virus to infect surrounding cells. It has been known since the 1957 that interferon production is a unique biological response to viral infection, which induces production against a broad variety of viral pathogens [75, 76]. Protection against viruses occurs through mechanisms of directly blocking the virus from replicating inside cells [77, 78], as well as stimulation of local immunity including antigen presenting cells [79-85], T cells [86-90], and natural killer (NK) cells [84, 91-98]. Interferon is viewed as the “First Responder” against global viral outbreaks [99]. Clinical trials and case reports support the efficacy of interferon therapy in deadly viral diseases including Ebola [100-102], Marburg [103], and Coronaviruses [104-108].

The dosages of interferons currently used for viral infections are extremely high, due in part, for need to administer systemically. To date, direct intra-pulmonary delivery has not been utilized. The current doses of interferon utilized appear to possess other side effects. Additionally, current interferons utilized are recombinant and do not represent the naturally occurring “symphony of cytokines” that occurs during a physiological anti-viral response.

In another embodiment, fibroblasts treated with TLR agonists, particularly TLR3 agonists, are utilized as a combination therapy with fibroblasts and/or with NK cells. In one embodiment, NK cells may be generated in vitro, as described below and admixed with TLR-3 activated fibroblasts in vitro enhance NK proliferation and cytotoxic activity in vitro. In other embodiments activated fibroblasts are administered in vivo together with NK cells. The cord blood may be utilized as a source of cytotoxic T cells and/or NK cells. In the NK cells obtained by a suitable preparation method, the pharmaceutical composition containing the NK cells and the cell therapy of the present disclosure, a solution for suspending or culturing living cells is, for example, a saline, a phosphate buffered saline (PBS), a medium, a serum or the like in general. The solution may comprise a carrier pharmaceutically acceptable as a pharmaceutical. The NK cells obtained by the preparation method of the present disclosure, the pharmaceutical composition containing the NK cells and the cell therapy of the present disclosure can be applied to treatment and/or prevention of various diseases having sensitivity to NK cells. Examples of such diseases include, but are not limited to, cancers and tumors such as an oral cancer, a gallbladder cancer, a cholangiocarcinoma, a lung cancer, a liver cancer, a colorectal cancer, a kidney cancer, a bladder cancer and leukemia, and infectious diseases caused by viruses, bacteria and the like. The pharmaceutical composition comprising the NK cells may contain, in addition to the NK cells prepared by the method of the present invention, an NK cell precursor, T cells, NKT cells, hematopoietic precursor cells and other cells in some cases. The cell therapy of the present invention may be practiced singly or in combination with surgical treatment, chemotherapy, radiation therapy or the like in some cases. In the cell therapy of the present disclosure, the NK cells expanded by the method of the present invention may be transplanted into a patient together with T cells and NKT cells in some cases. In the cell therapy of the present invention, the NK cells may be transplanted by, for example, intravenous, intraarterial, subcutaneous or intraperitoneal administration in some cases. In the method for preparing NK cells of the present invention, in the method for preparing the pharmaceutical composition of the present invention and in the cell therapy of the present invention, any of media such as, but not limited to, a KBM501 medium (Kohjin Bio Co., Ltd.), a CellGro SCGM medium (registered trademark, Cellgenix, Iwai Chemicals Company), a STEMLINE II (Sigma-Aldrich Co. LLC.), an X-VIVO15 medium (Lonza, Takara Bio Inc.), IMDM, MEM, DMEM and RPMI-1640 may be singly used as or blended in an appropriate ratio to be used as a medium for culturing cells in some cases. Besides, the media for culturing cells may be used with supplementation of at least one additional component selected from the group consisting of a serum, a serum albumin, an appropriate protein, a cytokine, an antibody, a compound and another component, which will be described below, in some cases.

The medium may be supplemented with an autologous serum of a subject, a human AB-type serum available from BioWhittaker Inc. or the like, or a donated human serum albumin available from Japanese Red Cross Society in some cases. The autologous serum and the human AB-type serum is supplemented preferably in a concentration of 1 to 10%, and the donated human serum albumin is supplemented preferably in a concentration of 1 to 10%. The subject may be a healthy person, or a patient having any of various diseases sensitive to NK cells. The medium may be supplemented with an appropriate protein, a cytokine, an antibody, a compound or another component as long as the effect of expanding NK cells is not impaired. The cytokine may be interleukin 2 (IL-2), interleukin 3 (IL-3), interleukin 7 (IL-7), interleukin 12 (IL-12), interleukin 15 (IL-15), interleukin 21 (IL-21), stem cell factor (SCF), thrombopoietin (TPO) and/or FMS-like tyrosine kinase 3 ligand (Flt3L) in some cases. The IL-2, IL-3, IL-7, IL-12, IL-15, IL-21, SCF, TPO and Flt3L may have a human amino acid sequence, and may be produced by a recombinant DNA technology from the safety viewpoint. The IL-15 may be used in a concentration of 0.1 to 100 ng/mL, such as in a concentration of 20 to 30 ng/mL, and including in a concentration of 25 ng/mL. The SCF may be used in a concentration of 2 to 100 ng/mL, such as in a concentration of 20 to 30 ng/mL, and including in a concentration of 25 ng/mL. The IL-7 may be used in a concentration of 0.5 to 100 ng/mL, such as in a concentration of 20 to 30 ng/mL, and including in a concentration of 25 ng/mL. The Flt3L may be used in a concentration of 1 to 100 ng/mL, such as in a concentration of 20 to 30 ng/mL, and including in a concentration of 25 ng/mL. The TPO may be used in a concentration of 1 to 100 ng/mL, such as in a concentration of 20 to 30 ng/mL, and including in a concentration of 25 ng/mL. Herein, the concentration of the IL-2 may be shown in Japanese Reference Unit (JRU) and International Unit (IU). Since 1 IU corresponds to approximately 0.622 JRU, 1750 JRU/mL corresponds to approximately 2813 IU/mL. The IL-2 may have a human amino acid sequence and may be produced by a recombinant DNA technology from the safety viewpoint. The IL-2 may be used in a concentration of 100 to 2900 IU/mL, such as in a concentration of 100 to 2813 IU/mL, including 2813 IU/mL. In the preparation method of the present disclosure and in the cell therapy of the present disclosure, in the step of expanding hematopoietic precursor cells, the cells are cultured in a medium supplemented with IL-15, SCF, IL-7 and Flt3L. The medium may be supplemented further with TPO in some cases. The medium may be replaced at any time after starting the cultivation as long as a desired number of NK cells can be obtained, and is preferably replaced every 3 to 5 days. In the expansion of the hematopoietic precursor cells, the cell growth rate is abruptly lowered in about 5 weeks. Therefore, the expansion of the hematopoietic precursor cells is conducted for about 5 weeks, namely, for 32, 33, 34, 35, 36, 37 or 38 days, after starting the cultivation. Thereafter, from the expanded hematopoietic precursor cells, NK cells are differentiation induced. In the step of differentially inducing NK cells, the cells are cultured in a medium supplemented with IL-2. The differentiation induction of NK cells is conducted for about 1 week, namely, for 5, 6, 7, 8 or 9 days. Here, cultivation conducted for n days under a given culturing condition refers to that the cultivation is conducted from a cultivation start date to n days after under the culturing condition, and means that transition to a next culturing condition or cell collection is performed n days after starting the cultivation. In the present invention, the hematopoietic precursor cells may be frozen during the expansion or after completing the expansion, and thawed in accordance with a time of transplantation into a patient to be used for the transplantation into the patient in some cases. The cells may be frozen and thawed by any of methods known to those skilled in the art. For freezing the cells, any of commercially available cryopreservation solutions is used in some cases.

In one embodiment of an expansion method, the culture vessel includes, but is not limited to, commercially available dishes, flasks, plates and multi-well plates. The culturing condition is not especially limited as long as the effect of expanding NK cells is not impaired, but a culturing condition of 37 C., 5% CO₂ and a saturated water vapor atmosphere is generally employed. Since the purpose of the present invention is to prepare a large amount of NK cells, it is advantageous that the time period of culturing the cells in the medium is longer because a larger amount of NK cells can be thus obtained. The culture period is not especially limited as long as the NK cells can be expanded to a desired number of cells.

The method and the production of the pharmaceutical composition of the present disclosure may be practiced under conditions complying with good manufacturing practices (GMP) for pharmaceuticals and quasi-pharmaceuticals. The cytotoxic activity of the NK cells thus prepared is evaluated by a method known to those skilled in the art. In general, the cytotoxic activity is quantitatively determined by incubating the NK cells (effector cells) and target cells labeled with a radioactive substance, a fluorescent dye or the like, and then measuring a radiation dose or a fluorescence intensity. The target cells may be K562 cells, acute myelogenous leukemia cells, or chronic myelogenous leukemia cells in some cases, but are not limited to these. The properties of the expanded NK cells may be checked by employing RT-PCR, solid phase hybridization, ELISA, Western blotting, immune precipitation, immunonephelometry, FACS, flow cytometry or the like in some cases. In the present disclosure, the collection and cryopreservation of an umbilical cord blood and/or adult blood cell tissue, the preparation of an autologous serum, the preparation of an umbilical cord blood and/or adult blood cell tissue, and mononuclear cells differentiation induced from pluripotent stem cells such as induced pluripotent stem cells, embryonic stem cells or adult stem cells, the preparation of hematopoietic precursor cells from the mononuclear cells, the measurement of the number of cells before and after the cultivation of the hematopoietic precursor cells, the measurement of a constituent ratio among NK cells, T cells and other cell types in the hematopoietic precursor cells before and after the cultivation, the calculation of the expansion factor of the NK cells, and the statistical analysis of a measurement error or significance may be practiced by any methods known to those skilled in the art.

The present disclosure provides cell therapy for an individual in need thereof. The cell therapy of the present disclosure includes a step of expanding hematopoietic precursor cells under a single culturing condition; and a step of differentially inducing the cells obtained in the expanding step into NK cells. In the cell therapy, a medium used in the step of expanding hematopoietic precursor cells under a single culturing condition may be supplemented with IL-15, SCF, IL-7 and Flt3L in some cases. In the cell therapy, the medium used in the step of expanding hematopoietic precursor cells under a single culturing condition may be supplemented further with TPO in some cases. In the cell therapy, the step of differentially inducing the NK cells may include culturing the expanded hematopoietic precursor cells under a culturing condition containing IL-2 in some cases. In the cell therapy, the medium used in each of the steps may be supplemented with a human AB-type serum and/or a human serum albumin. In the cell therapy, the hematopoietic precursor cells may be at least one of hematopoietic precursor cells selected from the group consisting of hematopoietic precursor cells contained in an umbilical cord blood and/or an adult blood cell tissue, hematopoietic precursor cells differentiation induced from induced pluripotent stem cells, embryonic stem cells and/or adult stem cells, and hematopoietic precursor cells directly converted from differentiated cells. In the cell therapy, the step of transplanting the NK cells into a patient may be a step of transplanting the NK cells together with other cells such as T cells or NKT cells in some cases. The cell therapy of the present invention may be employed for treating and/or preventing an infectious disease and/or a cancer.

Production of NK cells may comprise expanding a population of hematopoietic cells. During cell expansion, a plurality of hematopoietic cells within the hematopoietic cell population differentiate into NK cells. In one embodiment, provided herein is a method of producing a population of activated natural killer (NK) cells, comprising: (a) seeding a population of hematopoietic stem or progenitor cells in a first medium comprising interleukin-15 (IL-15) and, optionally, one or more of stem cell factor (SCF) and interleukin-7 (IL-7), wherein said IL-15 and optional SCF and IL-7 are not comprised within an undefined component of said medium, such that the population expands, and a plurality of hematopoietic stem or progenitor cells within said population of hematopoietic stem or progenitor cells differentiate into NK cells during said expanding; and (b) expanding the cells from step (a) in a second medium comprising interleukin-2 (IL-2), to produce a population of activated NK cells. In another embodiment, NK cells provided herein are produced by a two-step process of expansion/differentiation and maturation of NK cells. The first and second steps comprise culturing the cells in media with a unique combination of cellular factors. In certain embodiments, the process involves (a) culturing and expanding a population of hematopoietic cells in a first medium, wherein a plurality of hematopoietic stem or progenitor cells within the hematopoietic cell population differentiate into NK cells; and (b) expanding the NK cells from step (a) in a second medium, wherein the NK cells are further expanded and differentiated, and wherein the NK cells are maturated (e.g., activated or otherwise possessing cytotoxic activity). In certain embodiments, the method includes no intermediary steps between step (a) and (b), no additional culturing steps prior to step (a), and/or no additional steps (e.g., maturation step) after step (b). In certain embodiments, the methods provided herein comprises a first step of culturing and expanding a population of hematopoietic cells in a first medium, wherein a plurality of hematopoietic stem or progenitor cells within the hematopoietic cell population differentiate into NK cells. Without wishing to be bound by any parameter, mechanism or theory, culture of the hematopoietic cells as provided herein results in continuous expansion of the hematopoietic cells and differentiation of NK cells from said cells. In certain embodiments, hematopoietic cells, e.g., stem cells or progenitor cells, used in the methods provided herein are expanded and differentiated in the first step using a feeder layer. In other embodiments, hematopoietic cells, e.g., stem cells or progenitor cells, are expanded and differentiated in the first step without the use of a feeder layer. Feeder cell-independent expansion and differentiation of hematopoietic cells can take place in any container compatible with cell culture and expansion, e.g., flask, tube, beaker, dish, multiwell plate, bag or the like. In a specific embodiment, feeder cell-independent expansion of hematopoietic cells takes place in a bag, e.g., a flexible, gas-permeable fluorocarbon culture bag (for example, from American Fluoroseal). In a specific embodiment, the container in which the hematopoietic cells are expanded is suitable for shipping, e.g., to a site such as a hospital or military zone wherein the expanded NK cells are further expanded and differentiated.

The disclosure provides the utilization of fibroblasts together with adjuvants to reduce systemic cytokines that are toxic to the lung. In some embodiments cytokine levels are assessed and concentration of fibroblasts administered is adjusted. In some embodiments of the invention, fibroblasts are utilized together with mesenchymal stem cells, as well as with addition of adjuvants. Mesenchymal Stem Cells (MSCs) are potent immunomodulatory cells that recognize sites of injury, limit effector T cell reactions, and stimulate regulatory cell populations (i.e., T-regs) via growth factors, cytokines, and other mediators. Simultaneously, MSCs also stimulate local tissue regeneration via paracrine effects inducing angiogenic, anti-fibrotic and remodeling responses[109]. Consequently, MSCs-based therapy represent a viable treatment option for autoimmune conditions and other inflammatory disorders [110-115], yielding beneficial effects in models of autoimmune Type 1 Diabetes [116-122], Systemic Lupus Erythematosus, Autoimmune Encephalomyelitis [123], Multiple Sclerosis [124, 125], cardiac insufficiency [126, 127], and organ transplantation [128]. MSCs have been reported to inhibit inflammation and fibrosis in the lungs [129-132] and have been recently suggested as useful to treat patients with severe COVID-19 based on their effects preventing or attenuating the immunopathogenic cytokine storm [133-136]. MSCs can be easily derived in large numbers from the Umbilical Cord (UC) and can be rapidly expanded into clinically-relevant numbers. A population of UC-MSCs of interest was demonstrated to maintain its expansion capacity over 90 population doublings without cell senescence, while maintaining MSC characteristics and functions [137]. UC-MSC have demonstrated safety and efficacy in clinical trials, and have been safely administered across histocompatibility barriers.

### III. KITS

Any of the compositions described herein may be comprised in a kit. In a non-limiting example, the kit comprises fibroblasts and/or one or more adjuvants for fibroblasts. The kit may comprise any kind of adjuvants, including particular peptides (BPC-157; beta thymosine; and c) Pam₃CysSerLys₄), one or more activators of toll like receptors, hydroxychloroquine, resveratrol, losartan, azithromycin, a combination thereof, and so forth.

The kits may comprise a suitably aliquoted compositions of the present disclosure. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also may generally contain a second, third or other additional container into which the additional components may be separately placed. However, various combinations of components may be comprised in a vial. The kits of the present disclosure also will typically include a means for containing the compositions and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly considered. The compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

Irrespective of the number and/or type of containers, the kits of the disclosure may also comprise, and/or be packaged with, an instrument for assisting with the injection/administration and/or placement of the ultimate composition within the body of an animal. Such an instrument may be a syringe, pipette, forceps, and/or any such medically approved delivery vehicle.

### IV. EXAMPLES

The following examples are included to demonstrate particular embodiments of the invention. It should be appreciated by those of skill in the art that the techniques disclosed in the examples that follow represent techniques discovered by the inventor to function well in the practice of the embodiments of the disclosure, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the invention.

### Example 1

### Suppression of Pulmonary Leakage by Fibroblasts in Endotoxin ARDS Model

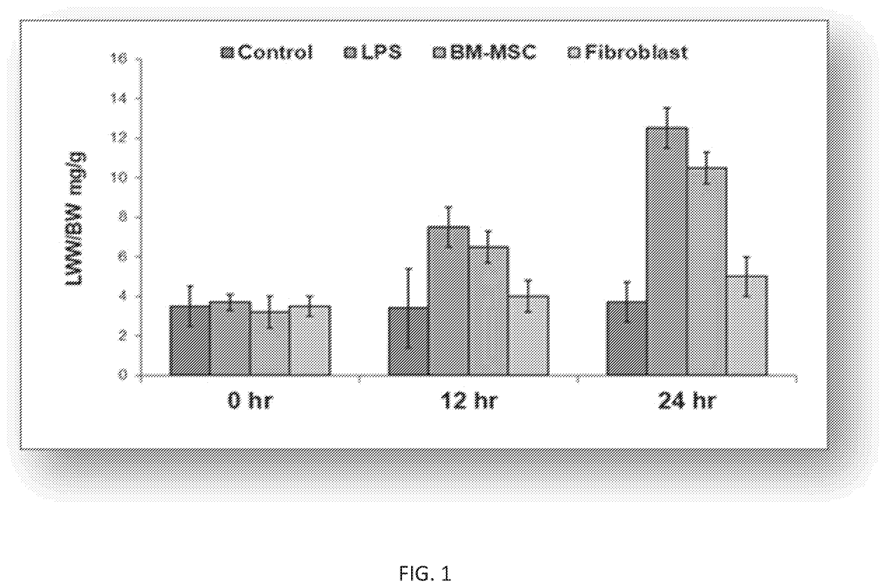

BALB/c female mice (10 per group) were intraperitoneally injected with 50 mg/kg pentobarbital. Lipopolysaccharides (LPS) (5 mg/kg) (Sigma-Aldrich) was delivered to the lungs through a tracheostomy. Fibroblasts (American Type Culture Collection; ATCC) were cultured for 48 hours in DMEM media with 10% fetal calf serum and selected for expression of CD73 using Magnetic Activated Cell Sorting (MACS). Bone marrow MSC (ATCC) were cultured in DMEM media with 10% fetal calf serum. Cells (500,000 cells in 150 μl PBS) were delivered via the tail vein 6 h after LPS administration. Animals were sacrificed after 24 h. Lung edema was assessed by quantify the ratio of lung wet weight to body weight ratios (LWW/BW). FIG. 1 shows suppression of the pulmonary leakage.

### Example 2

### Suppression of Neutrophil Infiltration by Fibroblasts in Endotoxin ARDS Model

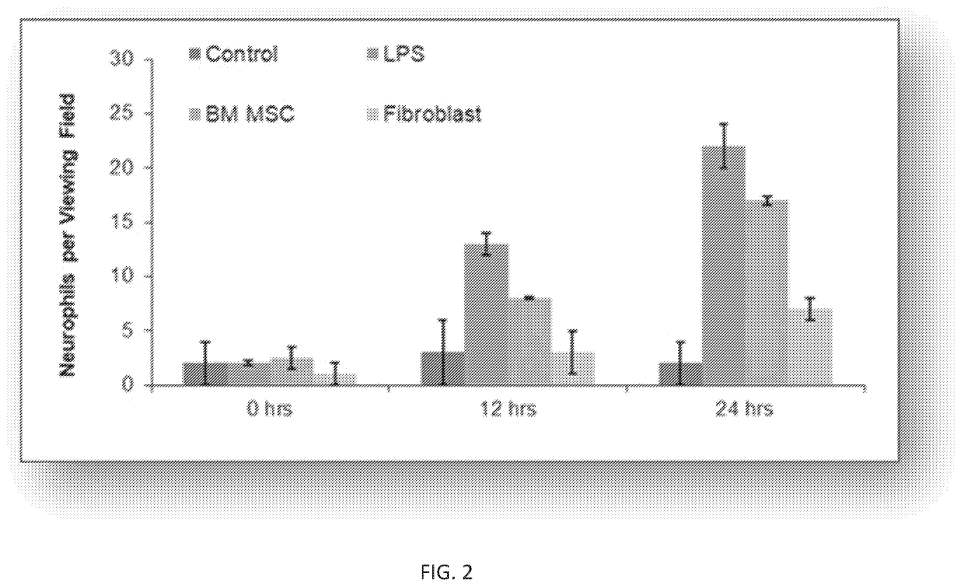

BALB/c female mice (10 per group) were intraperitoneally injected with 50 mg/kg pentobarbital. Lipopolysaccharides (LPS) (5 mg/kg) (Sigma-Aldrich) was delivered to the lungs through a tracheostomy. Fibroblasts (ATCC) were cultured for 48 hours in DMEM media with 10% fetal calf serum and selected for expression of CD73 using Magnetic Activated Cell Sorting (MACS). Bone marrow MSC (ATCC) were cultured in DMEM media with 10% fetal calf serum. Cells (500,000 cells in 150 μl PBS) were delivered via the tail vein 6 h after LPS administration. Animals were sacrificed after 24 h. Serial sections of lungs were made at 5 micro meter slices by cryostat and stated using H and E staining. Quantification of neutrophil number was performed by a blinded observer counting number of neutrophils per viewing field. FIG. 2 shows suppression of the neurophils.

### Example 3

### Suppression of TNF Alpha by Fibroblasts in Endotoxin ARDS Model

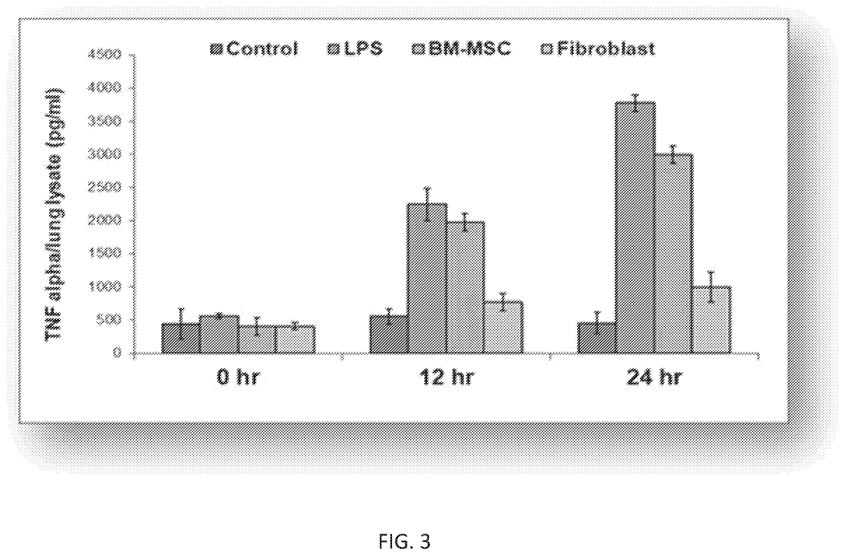

BALB/c female mice (10 per group) were intraperitoneally injected with 50 mg/kg pentobarbital. Lipopolysaccharides (LPS) (5 mg/kg) (Sigma-Aldrich) was delivered to the lungs through a tracheostomy. Fibroblasts (ATCC) were cultured for 48 hours in DMEM media with 10% fetal calf serum and selected for expression of CD73 using Magnetic Activated Cell Sorting (MACS). Bone marrow MSC (ATCC) were cultured in DMEM media with 10% fetal calf serum. Cells (500,000 cells in 150 μl PBS) were administered via the tail vein 6 h after LPS administration. Animals were sacrificed after 24 h. Quantification of TNF-alpha was performed using ELISA of lung lysate and expressed as pg/ml of lysate. FIG. 3 demonstrates suppression of TNF-alpha.

### Example 4

### Suppression of IL-6 by Fibroblasts in Endotoxin ARDS Model

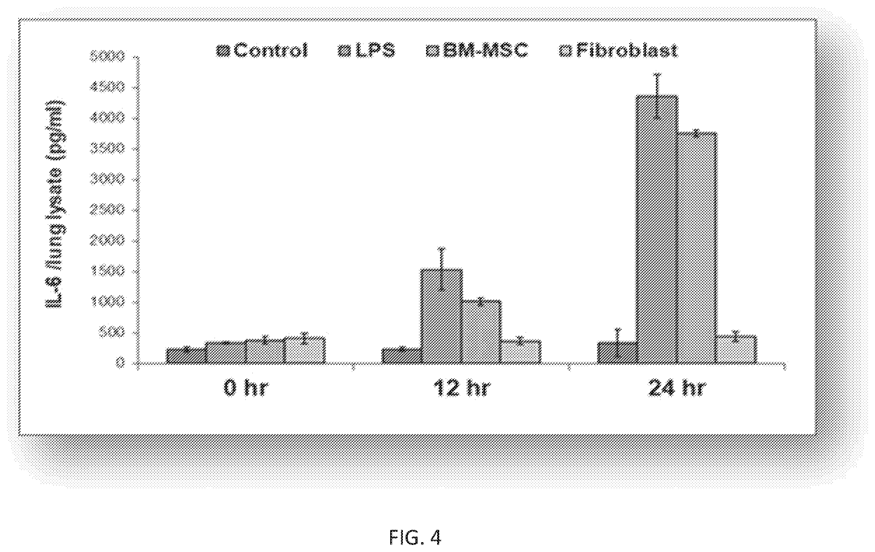

BALB/c female mice (10 per group) were intraperitoneally injected with 50 mg/kg pentobarbital. Lipopolysaccharides (LPS) (5 mg/kg) (Sigma-Aldrich) were delivered to the lungs through a tracheostomy. Fibroblasts (ATCC) were cultured for 48 hours in DMEM media with 10% fetal calf serum and selected for expression of CD73 using Magnetic Activated Cell Sorting (MACS). Bone marrow MSC (ATCC) were cultured in DMEM media with 10% fetal calf serum. Cells (500,000 cells in 150 μl PBS) were delivered via the tail vein 6 h after LPS administration. Animals were sacrificed after 24 h. Quantification of IL-6 was performed using ELISA of lung lysate and expressed as pg/ml of lysate. FIG. 4 shows suppression of IL-6.

### Example 5

### Hydroxychloroquine Anti-Inflammatory Efficacy Increased by Fibroblasts

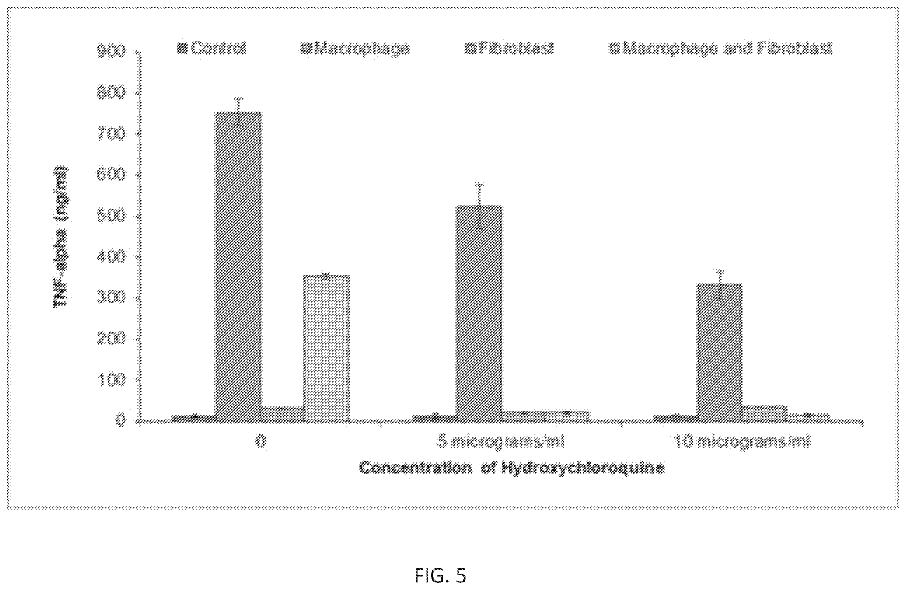

Endotoxin (LPS 1 ug/ml) was added to cultures of media alone (control), human peripheral blood mononuclear cell (PBMC) derived monocytes (positive control), fibroblasts (negative control, fibroblasts do not produce TNF alpha under these conditions), or the combination of cells at a 1:1 ratio. Hydroxychloroquine (Sigma Aldrich) was diluted at the concentrations indicated. Levels of tumor necrosis factor (TNF) were assessed by ELISA. In agreement with other studies, hydroxychloroquine suppressed production of TNF-alpha from macrophages. Interestingly, without hydroxychloroquine, fibroblasts suppressed macrophage TNF-alpha production by about 50%. When hydroxychloroquine was added, a synergistic inhibition of TNF-alpha was observed. FIG. 5 demonstrates the level of TNF-alpha as a function of concentration of hydroxychloroquine.

### Example 6

### Hydroxychloroquine Lung-Protecting Efficacy Increased by Fibroblasts

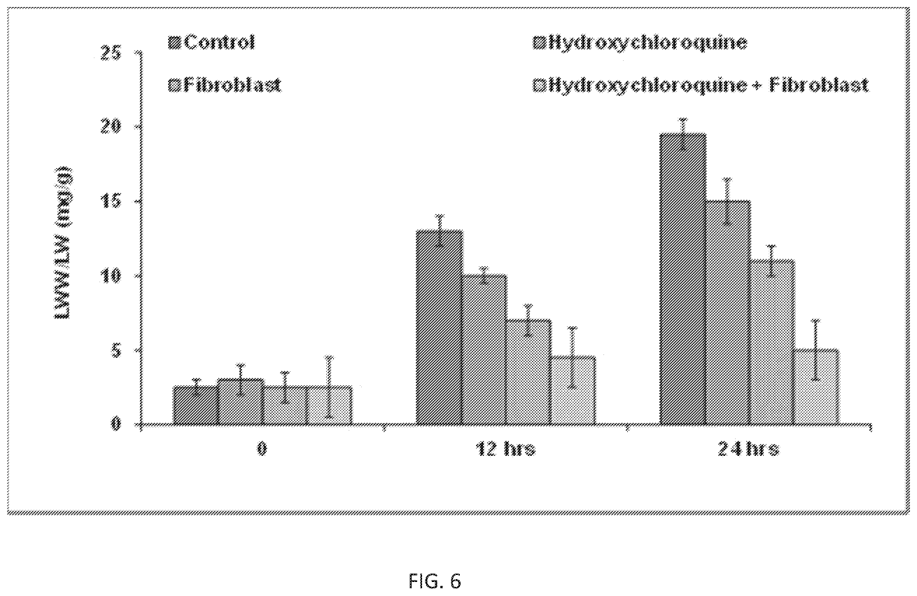

BALB/c female mice (10 per group) were intraperitoneally injected with 50 mg/kg pentobarbital. Lipopolysaccharides (LPS) (5 mg/kg) (Sigma-Aldrich) was delivered to the lungs through a tracheostomy. Fibroblasts (ATCC) were cultured for 48 hours in DMEM media with 10% fetal calf serum and selected for expression of CD73 using Magnetic Activated Cell Sorting (MACS). Bone marrow MSC (ATCC) were cultured in DMEM media with 10% fetal calf serum. Cells (500,000 cells in 150 μl PBS) were administered via the tail vein 6 h after LPS administration. Hydroxychloroquine was administered at 50 micrograms per mouse by gavage. Animals were sacrificed after 24 h. Lung edema was assessed by quantify the ratio of lung wet weight to body weight ratios (LWW/BW) (FIG. 6).

### REFERENCES

1. Zhu, N., et al., A Novel Coronavirus from Patients with Pneumonia in China, 2019. N Engl J Med, 2020. 382(8): p. 727-733.
2. Guo, Y. R., et al., The origin, transmission and clinical therapies on coronavirus disease 2019 (COVID-19) outbreak—an update on the status. Mil Med Res, 2020. 7(1): p. 11.
3. WHO, W.H.O., Coronavirus disease (COVID-19) outbreak 2020: p. https://www.who.int/emergencies/diseases/novel-coronavirus-2019.
4. Zhang, S., et al., Estimation of the reproductive number of Novel Coronavirus (COVID-19) and the probable outbreak size on the Diamond Princess cruise ship: A data-driven analysis. Int J Infect Dis, 2020.
5. Zhao, S., et al., Preliminary estimation of the basic reproduction number of novel coronavirus (2019-nCoV) in China, from 2019 to 2020: A data-driven analysis in the early phase of the outbreak. Int J Infect Dis, 2020. 92: p. 214-217.
6. Huang, C., et al., Clinical features of patients infected with 2019 novel coronavirus in Wuhan, China. Lancet, 2020. 395(10223): p. 497-506.
7. Wang, D., et al., Clinical Characteristics of 138 Hospitalized Patients With 2019 Novel Coronavirus-Infected Pneumonia in Wuhan, China. JAMA, 2020.
8. Chen, N., et al., Epidemiological and clinical characteristics of 99 cases of 2019 novel coronavirus pneumonia in Wuhan, China: a descriptive study. Lancet, 2020. 395(10223): p. 507-513.
9. Grasselli, G., A. Pesenti, and M. Cecconi, Critical Care Utilization for the COVID-19 Outbreak in Lombardy, Italy: Early Experience and Forecast During an Emergency Response. JAMA, 2020.
10. Wu, C., et al., Risk Factors Associated With Acute Respiratory Distress Syndrome and Death in Patients With Coronavirus Disease 2019 Pneumonia in Wuhan, China. JAMA Intern Med, 2020.
11. Shi, H., et al., Radiological findings from 81 patients with COVID-19 pneumonia in Wuhan, China: a descriptive study. Lancet Infect Dis, 2020.
12. Xu, Z., et al., Pathological findings of COVID-19 associated with acute respiratory distress syndrome. Lancet Respir Med, 2020.
13. Tian, S., et al., Pulmonary pathology of early phase 2019 novel coronavirus (COVID-19) pneumonia in two patients with lung cancer. J Thorac Oncol, 2020.
14. Buttner, S., et al., Extracorporeal virus elimination for the treatment of severe Ebola virus disease first experience with lectin affinity plasmapheresis. Blood Purif, 2014. 38(3-4): p. 286-91.
15. Dal Nogare, A. R., Adult respiratory distress syndrome. Am J Med Sci, 1989. 298(6): p. 413-30.
16. Suter, P. M., et al., High bronchoalveolar levels of tumor necrosis factor and its inhibitors, interleukin-1, interferon, and elastase, in patients with adult respiratory distress syndrome after trauma, shock, or sepsis. Am Rev Respir Dis, 1992. 145(5): p. 1016-22.
17. Larrick, J. W., et al., Recombinant tumor necrosis factor causes activation of human granulocytes. Blood, 1987. 69(2): p. 640-4.
18. Siler, T. M., et al., Immunoreactive interleukin-1 in bronchoalveolar lavage fluid of high-risk patients and patients with the adult respiratory distress syndrome. Exp Lung Res, 1989. 15(6): p. 881-94.
19. Jacobs, R. F., et al., Elevated interleukin-1 release by human alveolar macrophages during the adult respiratory distress syndrome. Am Rev Respir Dis, 1989. 140(6): p. 1686-92.
20. Zhao, M. W., D. J. Sun, and D. L. Ma, [Protective effect of interleukin-1 receptor antagonist on oleic acid-induced lung injury]. Zhonghua Nei Ke Za Zhi, 1994. 33(3): p. 158-61.
21. Fisher, C. J., Jr., et al., Role of interleukin-1 and the therapeutic potential of interleukin-1 receptor antagonist in sepsis. Circ Shock, 1994. 44(1): p. 1-8.
22. Repine, J. E., Interleukin-1-mediated acute lung injury and tolerance to oxidative injury. Environ Health Perspect, 1994. 102 Suppl 10: p. 75-8.
23. Patton, L. M., et al., Interleukin-1 beta-induced neutrophil recruitment and acute lung injury in hamsters. Inflammation, 1995. 19(1): p. 23-9.
24. Bellomo, R., The cytokine network in the critically ill. Anaesth Intensive Care, 1992. 20(3): p. 288-302.
25. Ghezzi, P., et al., Hypoxia increases production of interleukin-1 and tumor necrosis factor by human mononuclear cells. Cytokine, 1991. 3(3): p. 189-94.
26. Eisenhut, T., et al., Prostacyclin analogs suppress the synthesis of tumor necrosis factor-alpha in LPS-stimulated human peripheral blood mononuclear cells. Immunopharmacology, 1993. 26(3): p. 259-64.
27. Ghochikyan, A., et al., Targeting TLR-4 with a novel pharmaceutical grade plant derived agonist, Immunomax(R), as a therapeutic strategy for metastatic breast cancer. J Transl Med, 2014. 12: p. 322.
28. Oronsky, B., et al., RRx-001: a systemically non-toxic M2-to-M1 macrophage stimulating and prosensitizing agent in Phase II clinical trials. Expert Opin Investig Drugs, 2017. 26(1): p. 109-119.
29. Lee, C., et al., Melittin suppresses tumor progression by regulating tumor-associated macrophages in a Lewis lung carcinoma mouse model. Oncotarget, 2017. 8(33): p. 54951-54965.
30. Zhang, Q., et al., Clinical Effects of CpG-Based Treatment on the Efficacy of Hepatocellular Carcinoma by Skewing Polarization Toward M1 Macrophage from M2. Cancer Biother Radiopharm, 2017. 32(6): p. 215-219.
31. Sato, T., et al., Intrapulmonary Delivery of CpG Microparticles Eliminates Lung Tumors. Mol Cancer Ther, 2015. 14(10): p. 2198-205.
32. Chiang, C. F., et al., Metformin-treated cancer cells modulate macrophage polarization through AMPK-NF-kappaB signaling. Oncotarget, 2017. 8(13): p. 20706-20718.
33. Kang, H., et al., Puerarin inhibits M2 polarization and metastasis of tumor-associated macrophages from NSCLC xenograft model via inactivating MEK/ERK 1/2 pathway. Int J Oncol, 2017. 50(2): p. 545-554.
34. Jia, X., et al., Emodin suppresses pulmonary metastasis of breast cancer accompanied with decreased macrophage recruitment and M2 polarization in the lungs. Breast Cancer Res Treat, 2014. 148(2): p. 291-302.
35. Xue, N., et al., Chlorogenic acid inhibits glioblastoma growth through repolarizating macrophage from M2 to M1 phenotype. Sci Rep, 2017. 7: p. 39011.
36. Sloan, E. K., et al., The sympathetic nervous system induces a metastatic switch in primary breast cancer. Cancer Res, 2010. 70(18): p. 7042-52.
37. Liu, B., et al., Polarization of M1 tumor associated macrophage promoted by the activation of TLR3 signal pathway. Asian Pac J Trop Med, 2016. 9(5): p. 484-8.
38. Liu, Q., et al., NMAAP1 Expressed in BCG-Activated Macrophage Promotes M1 Macrophage Polarization. Mol Cells, 2015. 38(10): p. 886-94.
39. Liu, Y., et al., Polysaccharide Agaricus blazei Murill stimulates myeloid derived suppressor cell differentiation from M2 to M1 type, which mediates inhibition of tumour immune-evasion via the Toll-like receptor 2 pathway. Immunology, 2015. 146(3): p. 379-91.
40. Yang, Y., et al., LPS converts Gr-1(+)CD115(+) myeloid-derived suppressor cells from M2 to M1 via P38 MAPK. Exp Cell Res, 2013. 319(12): p. 1774-83.
41. Sanchez-Quesada, C., A. Lopez-Biedma, and J. J. Gaforio, Maslinic Acid enhances signals for the recruitment of macrophages and their differentiation to m1 state. Evid Based Complement Alternat Med, 2015. 2015: p. 654721.
42. Dominguez-Soto, A., et al., Intravenous immunoglobulin promotes antitumor responses by modulating macrophage polarization. J Immunol, 2014. 193(10): p. 5181-9.
43. Yin, Y., et al., Phosphatidylserine-targeting antibody induces M1 macrophage polarization and promotes myeloid-derived suppressor cell differentiation. Cancer Immunol Res, 2013. 1(4): p. 256-68.
44. Deng, R., et al., Dimethyl Sulfoxide Suppresses Mouse 4T1 Breast Cancer Growth by Modulating Tumor-Associated Macrophage Differentiation. J Breast Cancer, 2014. 17(1): p. 25-32.
45. Mitsuhashi, A., et al., Surfactant protein A suppresses lung cancer progression by regulating the polarization of tumor-associated macrophages. Am J Pathol, 2013. 182(5): p. 1843-53.
46. Coscia, M., et al., Zoledronic acid repolarizes tumour-associated macrophages and inhibits mammary carcinogenesis by targeting the mevalonate pathway. J Cell Mol Med, 2010. 14(12): p. 2803-15.
47. Eriksson, F., et al., Tumor-specific bacteriophages induce tumor destruction through activation of tumor-associated macrophages. J Immunol, 2009. 182(5): p. 3105-11.
48. Zhang, J., et al., Macrophage activation by edible mushrooms is due to the collaborative interaction of toll-like receptor agonists and dectin-1b activating beta glucans derived from colonizing microorganisms. Food Funct, 2019. 10(12): p. 8208-8217.
49. Wang, Y., et al., Adjuvant effect of the novel TLR1/TLR2 agonist Diprovocim synergizes with anti-PD-L1 to eliminate melanoma in mice. Proc Natl Acad Sci USA, 2018. 115(37): p. E8698-E8706.
50. Qian, J., et al., TLR2 Promotes Glioma Immune Evasion by Downregulating MHC Class II Molecules in Microglia. Cancer Immunol Res, 2018. 6(10): p. 1220-1233.
51. Lancioni, C. L., et al., Mycobacterium tuberculosis lipoproteins directly regulate human memory CD4(+) T cell activation via Toll-like receptors 1 and 2. Infect Immun, 2011. 79(2): p. 663-73.
52. Su, H., et al., Recombinant Lipoprotein Rv1016c Derived from Mycobacterium tuberculosis Is a TLR-2 Ligand that Induces Macrophages Apoptosis and Inhibits MHC II Antigen Processing. Front Cell Infect Microbiol, 2016. 6: p. 147.
53. Abe, T., et al., Key role of CD36 in Toll-like receptor 2 signaling in cerebral ischemia. Stroke, 2010. 41(5): p. 898-904.
54. Doni, A., et al., Regulation of PTX3, a key component of humoral innate immunity in human dendritic cells: stimulation by IL-10 and inhibition by IFN-gamma. J Leukoc Biol, 2006. 79(4): p. 797-802.
55. Seya, T., Y. Takeda, and M. Matsumoto, A Toll-like receptor 3 (TLR3) agonist ARNAX for therapeutic immunotherapy. Adv Drug Deliv Rev, 2019. 147: p. 37-43.
56. Roten, R., et al., Plasma levels of tumor necrosis factor in the adult respiratory distress syndrome. Am Rev Respir Dis, 1991. 143(3): p. 590-2.
57. Sheng, W. H., et al., Clinical manifestations and inflammatory cytokine responses in patients with severe acute respiratory syndrome. J Formos Med Assoc, 2005. 104(10): p. 715-23.
58. Romaschin, A. D., et al., Systemic phospholipase A2 and cachectin levels in adult respiratory distress syndrome and multiple-organ failure. Clin Biochem, 1992. 25(1): p. 55-60.
59. Koh, Y., et al., Tumor necrosis factor induced acute lung leak in rats: less than with interleukin-1. Inflammation, 1996. 20(5): p. 461-9.
60. Dunican, A. L., et al., TNFalpha-induced suppression of PMN apoptosis is mediated through interleukin-8 production. Shock, 2000. 14(3): p. 284-8; discussion 288-9.
61. Negrier, M. S., et al., Phase I trial of recombinant interleukin-2 followed by recombinant tumor necrosis factor in patients with metastatic cancer. J Immunother (1991), 1992. 11(2): p. 93-102.
62. Malemud, C. J., Recent advances in neutralizing the IL-6 pathway in arthritis. Open Access Rheumatol, 2009. 1: p. 133-150.
63. Romagnani, S., Human Th17 cells. Arthritis Res Ther, 2008. 10(2): p. 206.
64. Romagnani, S., et al., Properties and origin of human Th17 cells. Mol Immunol, 2009. 47(1): p. 3-7.
65. Kimura, A. and T. Kishimoto, IL-6: regulator of Treg/Th17 balance. Eur J Immunol, 2010. 40(7): p. 1830-5.
66. Yu, Z. X., et al., The ratio of Th17/Treg cells as a risk indicator in early acute respiratory distress syndrome. Crit Care, 2015. 19: p. 82.
67. Mock, J. R., et al., Foxp3+ regulatory T cells promote lung epithelial proliferation. Mucosal Immunol, 2014. 7(6): p. 1440-51.
68. Kimura, A. and T. Kishimoto, Th17 cells in inflammation. Int Immunopharmacol, 2011. 11(3): p. 319-22.
69. Meduri, G. U., et al., Persistent elevation of inflammatory cytokines predicts a poor outcome in ARDS. Plasma IL-1 beta and IL-6 levels are consistent and efficient predictors of outcome over time. Chest, 1995. 107(4): p. 1062-73.
70. Hui, L., et al., Higher serum procalcitonin and IL-6 levels predict worse diagnosis for acute respiratory distress syndrome patients with multiple organ dysfunction. Int J Clin Exp Pathol, 2017. 10(7): p. 7401-7407.
71. Swaroopa, D., et al., Association of serum interleukin-6, interleukin-8, and Acute Physiology and Chronic Health Evaluation II score with clinical outcome in patients with acute respiratory distress syndrome. Indian J Crit Care Med, 2016. 20(9): p. 518-25.
72. Bime, C., et al., Development of a biomarker mortality risk model in acute respiratory distress syndrome. Crit Care, 2019. 23(1): p. 410.
73. Spadaro, S., et al., Biomarkers for Acute Respiratory Distress syndrome and prospects for personalised medicine. J Inflamm (Lond), 2019. 16: p. 1.
74. Channappanavar, R. and S. Perlman, Pathogenic human coronavirus infections: causes and consequences of cytokine storm and immunopathology. Semin Immunopathol, 2017. 39(5): p. 529-539.
75. Isaacs, A. and J. Lindenmann, Virus interference. I. The interferon. Proc R Soc Lond B Biol Sci, 1957. 147(927): p. 258-67.
76. Isaacs, A., J. Lindenmann, and R. C. Valentine, Virus interference. II. Some properties of interferon. Proc R Soc Lond B Biol Sci, 1957. 147(927): p. 268-73.
77. Johnson, H. M., Differentiation of the immunosuppressive and antiviral effects of interferon. Cell Immunol, 1978. 36(2): p. 220-30.
78. Minagawa, H., et al., Protective effect of recombinant murine interferon beta against mouse hepatitis virus infection. Antiviral Res, 1987. 8(2): p. 85-95.
79. van Seventer, J. M., T. Nagai, and G. A. van Seventer, Interferon-beta differentially regulates expression of the IL-12 family members p35, p40, p19 and EBI3 in activated human dendritic cells. J Neuroimmunol, 2002. 133(1-2): p. 60-71.
80. Hermanrud, C., et al., Different Interferon Beta Preparations Induce the Same Qualitative Immune Response in Human Skin. J Interferon Cytokine Res, 2019. 39(5): p. 302-313.
81. Kadowaki, N., et al., Natural interferon alpha/beta-producing cells link innate and adaptive immunity. J Exp Med, 2000. 192(2): p. 219-26.
82. Cremer, I., et al., Inhibition of human immunodeficiency virus transmission to CD4+ T cells after gene transfer of constitutively expressed interferon beta to dendritic cells. Hum Gene Ther, 2000. 11(12): p. 1695-703.
83. Izuma, M., et al., In vitro cytokine production of peripheral blood mononuclear cells in response to HCV core antigen stimulation during interferon-beta treatment and its relevance to sCD8 and sCD30. Hepatol Res, 2000. 18(3): p. 218-229.
84. Dalod, M., et al., Interferon alpha/beta and interleukin 12 responses to viral infections: pathways regulating dendritic cell cytokine expression in vivo. J Exp Med, 2002. 195(4): p. 517-28.
85. Honda, K., et al., Selective contribution of IFN-alpha/beta signaling to the maturation of dendritic cells induced by double-stranded RNA or viral infection. Proc Natl Acad Sci USA, 2003. 100(19): p. 10872-7.
86. Byrnes, A. A., et al., Type I interferons and IL-12: convergence and cross-regulation among mediators of cellular immunity. Eur J Immunol, 2001. 31(7): p. 2026-34.
87. McNally, J. M., et al., Attrition of bystander CD8 T cells during virus-induced T-cell and interferon responses. J Virol, 2001. 75(13): p. 5965-76.
88. Biron, C. A., Role of early cytokines, including alpha and beta interferons (IFN-alpha/beta), in innate and adaptive immune responses to viral infections. Semin Immunol, 1998. 10(5): p. 383-90.
89. Sareneva, T., et al., Influenza A virus-induced IFN-alpha/beta and IL-18 synergistically enhance IFN-gamma gene expression in human T cells. J Immunol, 1998. 160(12): p. 6032-8.
90. Brown, J. L., J. Barsoum, and X. Q. Qin, CD4+ T helper cell-independent antitumor response mediated by murine IFN-beta gene delivery in immunocompetent mice. J Interferon Cytokine Res, 2002. 22(6): p. 719-28.
91. Johnson, H. M. and S. Baron, Interferon: effects on the immune response and the mechanism of activation of the cellular response. CRC Crit Rev Biochem, 1976. 4(2): p. 203-27.
92. Kuroki, M., et al., Antitumor effect of recombinant murine interferon-beta against mouse malignant glioma. J Interferon Res, 1987. 7(3): p. 301-11.
93. Markovic, S. N., P. R. Knight, and D. M. Murasko, Inhibition of interferon stimulation of natural killer cell activity in mice anesthetized with halothane or isoflurane. Anesthesiology, 1993. 78(4): p. 700-6.
94. Zdravkovic, M., et al., In vitro cytotoxic activity of cord blood NK cells against herpes simplex virus type-1 infected purified human term villous cytotrophoblast. Viral Immunol, 1994. 7(3): p. 133-40.
95. Biron, C. A., Natural killer cell regulation during viral infection. Biochem Soc Trans, 1997. 25(2): p. 687-90.
96. Weinberg, A., L. Zhang, and R. M. Tuder, NK recognition of cytomegalovirus-infected endothelial cells depends on viral replication and MHC class I expression. Viral Immunol, 1996. 9(2): p. 131-40.
97. Sato, K., et al., Antiviral response by natural killer cells through TRAIL gene induction by IFN-alpha/beta. Eur J Immunol, 2001. 31(11): p. 3138-46.
98. Nguyen, K. B., et al., Coordinated and distinct roles for IFN-alpha beta, IL-12, and IL-15 regulation of NK cell responses to viral infection. J Immunol, 2002. 169(8): p. 4279-87.
99. Wang, B. X. and E. N. Fish, Global virus outbreaks: Interferons as 1st responders. Semin Immunol, 2019. 43: p. 101300.
100. Konde, M. K., et al., Interferon beta-1a for the treatment of Ebola virus disease: A historically controlled, single-arm proof-of-concept trial. PLoS One, 2017. 12(2): p. e0169255.
101. Wrensch, F., et al., Interferon-Induced Transmembrane Protein-Mediated Inhibition of Host Cell Entry of Ebolaviruses. J Infect Dis, 2015. 212 Suppl 2: p. S210-8.
102. Shuchman, M., Could interferon help treat Ebola? CMAJ, 2014. 186(16): p. 1204.
103. Smith, L. M., et al., Interferon-beta therapy prolongs survival in rhesus macaque models of Ebola and Marburg hemorrhagic fever. J Infect Dis, 2013. 208(2): p. 310-8.
104. Al-Tawfiq, J. A., et al., Ribavirin and interferon therapy in patients infected with the Middle East respiratory syndrome coronavirus: an observational study. Int J Infect Dis, 2014. 20: p. 42-6.
105. Faure, E., et al., Distinct immune response in two MERS-CoV-infected patients: can we go from bench to bedside? PLoS One, 2014. 9(2): p. e88716.
106. Khalid, M., et al., Ribavirin and interferon-alpha2b as primary and preventive treatment for Middle East respiratory syndrome coronavirus: a preliminary report of two cases. Antivir Ther, 2015. 20(1): p. 87-91.
107. Kim, U. J., et al., Combination therapy with lopinavir/ritonavir, ribavirin and interferon-alpha for Middle East respiratory syndrome. Antivir Ther, 2016. 21(5): p. 455-9.
108. Mayer, K., et al., Fatal outcome of human coronavirus NL63 infection despite successful viral elimination by IFN-alpha in a patient with newly diagnosed ALL. Eur J Haematol, 2016. 97(2): p. 208-210.
109. Caplan, A. I. and D. Correa, The MSC: an injury drugstore. Cell Stem Cell, 2011. 9(1): p. 11-5.
110. Ge, W., et al., Regulatory T-cell generation and kidney allograft tolerance induced by mesenchymal stem cells associated with indoleamine 2,3-dioxygenase expression. Transplantation, 2010. 90(12): p. 1312-20.
111. Bartholomew, A., et al., Mesenchymal stem cells suppress lymphocyte proliferation in vitro and prolong skin graft survival in vivo. Experimental hematology, 2002. 30(1): p. 42-8.
112. Casiraghi, F., et al., Pretransplant infusion of mesenchymal stem cells prolongs the survival of a semiallogeneic heart transplant through the generation of regulatory T cells. Journal of immunology, 2008. 181(6): p. 3933-46.
113. Ge, W., et al., Infusion of mesenchymal stem cells and rapamycin synergize to attenuate alloimmune responses and promote cardiac allograft tolerance. American journal of transplantation: official journal of the American Society of Transplantation and the American Society of Transplant Surgeons, 2009. 9(8): p. 1760-72.
114. Uccelli, A., A. Laroni, and M. S. Freedman, Mesenchymal stem cells for the treatment of multiple sclerosis and other neurological diseases. Lancet neurology, 2011. 10(7): p. 649-56.
115. Ge, W., et al., Infusion of mesenchymal stem cells and rapamycin synergize to attenuate alloimmune responses and promote cardiac allograft tolerance. Am J Transplant, 2009. 9(8): p. 1760-72.
116. Perez-Simon, J. A., et al., Mesenchymal stem cells expanded in vitro with human serum for the treatment of acute and chronic graft-versus-host disease: results of a phase I/II clinical trial. Haematologica, 2011. 96(7): p. 1072-6.
117. Garcia-Olmo, D., et al., A phase I clinical trial of the treatment of Crohn's fistula by adipose mesenchymal stem cell transplantation. Dis Colon Rectum, 2005. 48(7): p. 1416-23.
118. Sun, L., et al., Mesenchymal stem cell transplantation reverses multiorgan dysfunction in systemic lupus erythematosus mice and humans. Stem Cells, 2009. 27(6): p. 1421-32.
119. Dhere, T., et al., The safety of autologous and metabolically fit bone marrow mesenchymal stromal cells in medically refractory Crohn's disease—a phase 1 trial with three doses. Aliment Pharmacol Ther, 2016. 44(5): p. 471-81.
120. Carlsson, P. O., et al., Preserved beta-cell function in type 1 diabetes by mesenchymal stromal cells. Diabetes, 2015. 64(2): p. 587-92.
121. Cal, J., et al., Umbilical Cord Mesenchymal Stromal Cell With Autologous Bone Marrow Cell Transplantation in Established Type 1 Diabetes: A Pilot Randomized Controlled Open-Label Clinical Study to Assess Safety and Impact on Insulin Secretion. Diabetes Care, 2016. 39(1): p. 149-57.
122. Hu, J., et al., Long term effects of the implantation of Wharton's jelly-derived mesenchymal stem cells from the umbilical cord for newly-onset type 1 diabetes mellitus. Endocr J, 2013. 60(3): p. 347-57.
123. Morando, S., et al., The therapeutic effect of mesenchymal stem cell transplantation in experimental autoimmune encephalomyelitis is mediated by peripheral and central mechanisms. Stem cell research & therapy, 2012. 3(1): p. 3.
124. Hedayatpour, A., et al., Promotion of remyelination by adipose mesenchymal stem cell transplantation in a cuprizone model of multiple sclerosis. Cell journal, 2013. 15(2): p. 142-51.
125. Al Jumah, M. A. and M. H. Abumaree, The immunomodulatory and neuroprotective effects of mesenchymal stem cells (MSCs) in experimental autoimmune encephalomyelitis (EAE): a model of multiple sclerosis (MS). Int J Mol Sci, 2012. 13(7): p. 9298-331.
126. Bartolucci, J., et al., Safety and Efficacy of the Intravenous Infusion of Umbilical Cord Mesenchymal Stem Cells in Patients With Heart Failure: A Phase 1/2 Randomized Controlled Trial (RIMECARD Trial [Randomized Clinical Trial of Intravenous Infusion Umbilical Cord Mesenchymal Stem Cells on Cardiopathy]). Circ Res, 2017. 121(10): p. 1192-1204.
127. Tuma, J., et al., RESCUE-HF Trial: Retrograde Delivery of Allogeneic Umbilical Cord Lining Subepithelial Cells in Patients With Heart Failure. Cell Transplant, 2016. 25(9): p. 1713-1721.
128. Franquesa, M., et al., Mesenchymal Stem Cells in Solid Organ Transplantation (MiSOT) Fourth Meeting: lessons learned from first clinical trials. Transplantation, 2013. 96(3): p. 234-8.
129. Moroncini, G., et al., Mesenchymal stromal cells from human umbilical cord prevent the development of lung fibrosis in immunocompetent mice. PLoS One, 2018. 13(6): p. e0196048.
130. Zhang, C., et al., Clinical observation of umbilical cord mesenchymal stem cell treatment of severe idiopathic pulmonary fibrosis: A case report. Exp Ther Med, 2017. 13(5): p. 1922-1926.
131. Moodley, Y., et al., Anti-inflammatory effects of adult stem cells in sustained lung injury: a comparative study. PLoS One, 2013. 8(8): p. e69299.
132. Harrell, C. R., et al., Mesenchymal Stem Cell-Based Therapy of Inflammatory Lung Diseases: Current Understanding and Future Perspectives. Stem Cells Int, 2019. 2019: p. 4236973.
133. Leng, Z., et al., Transplantation of ACE2-mesenchymal stem cells improves the outcome of patients with COVID-19 pneumonia. ChinaXiv, 2020.
134. Liang, B., et al., Clinical remission of a critically ill COVID-19 patient treated by human umbilical cord mesenchymal stem cells. ChinaXiv, 2020.
135. Zikuan Leng, R. Z., Wei Hou, Yingmei Feng, Yanlei Yang, Qin Han, Guangliang Shan, Fanyan Meng, Dongshu Du, Shihua Wang, Junfen Fan, Wenjing Wang, Luchan Deng, Hongbo Shi, Hongjun Li, Zhongjie Hu, Fengchun Zhang, Jinming Gao, Hongjian Liu, Xiaoxia Li, Yangyang Zhao, Kan Yin, Xijing He, Zhengchao Gao, Yibin Wang, Bo Yang, Ronghua Jin, Ilia Stambler, Lee Wei Lim, Huanxing Su, Alexey Moskalev, Antonio Cano, Sasanka Chakrabarti, Kyung-Jin Min, Georgina Ellison-Hughes, Calogero Caruso, Kunlin Jin, Robert Chunhua Zhao, Transplantation of ACE2<sup>-</sup> Mesenchymal Stem Cells Improves the Outcome of Patients with COVID-19 Pneumonia. Aging and disease, 2020. 11(2): p. 216-228.
136. Peng, Z., Umbilical Cord(UC)-Derived Mesenchymal Stem Cells(MSCs) Treatment for the 2019-novel Coronavirus(nCOV) Pneumonia.
137. Patel A N, V. V., Raveloo P, Bull D A, Mesenchymal Stem Cell Population Isoalted From the Subepithelial Layer of Umbilical Cord Tissue. Cell Transplantation, 2013: p. 513-519.
138. Baumel, B. A Phase I, Prospective, Open-label Trial to Evaluate the Safety, Tolerability and Exploratory Outcomes of Multiple Allogeneic Human Mesenchymal Stem Cells (HMSC) Infusions in Patients With Mild to Moderate Alzheimer's Disease. [cited 2020 Mar. 9.]

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A method of treating or preventing a viral infection and/or acute respiratory distress syndrome (ARDS) in an individual, comprising administering to the individual a therapeutically effective amount of one or more adjuvants and a therapeutically effective amount of fibroblasts and/or fibroblast-derived products.

2. The method of claim 1, wherein the viral infection is from adenovirus, alphavirus, BK virus, Bocavirus, calicivirus, coronavirus, cytomegalovirus, distemper virus, Ebola virus, enterovirus, Epstein Barr virus, Varicella-zoster virus, flavivirus, hepatitis virus (AE), herpesvirus, infectious peritonitis virus, influenza virus, John Cunningham virus, leukemia virus, Lymphocytic choriomeningitis virus, Marburg virus, metapneumovirus, norovirus, orthomyxovirus, papilloma virus, parainfluenza virus, paramyxovirus, parvovirus, pestivirus, picorna virus, pox virus, rabies virus, reovirus, retrovirus, rhinovirus, Respiratory Syncytial Virus, rotavirus, West Nile virus, Zika virus, a virus that causes the common cold, or a virus that causes cancer.

3. The method of claim 1 or 2, wherein the viral infection is a coronavirus infection.

4. The method of claim 3, wherein the coronavirus infection is from SARS-CoV-2.

5. The method of any one of claims 1-4, wherein said adjuvants comprise one or more peptides.

6. The method of claim 5, wherein said peptides are selected from the group consisting of a) BPC-157; b) beta thymosine; c) Pam₃CysSerLys₄ (SEQ ID NO: 3); d) functional derivatives thereof and e) a combination thereof.

7. The method of any one of claims 1-6, wherein said adjuvants comprise one or more activators of one or more toll like receptors.

8. The method of claim 7, wherein said toll like receptor comprises toll like receptor 2.

9. The method of claim 7 or 8, wherein said activator of toll like receptor 2 is selected from the group consisting of a) PAM2CSK4 (SEQ ID NO: 4); b) beta glucan; c) water insoluble fractions of medicinal mushrooms (Lentinula edodes, Grifola frondosa, Hypsizygus marmoreus varieties, Flammulina velutipes); d) Diprovocim; e) HSPA4; f) HSPA5; g) HSPA9; h) HSPA13; i) HSPD1; j) VCAN; k) Lipoproteins LprG and LpqH; l) MTB lipoprotein Rv1016c; m) HKLM n) FSL-1; and o) a combination thereof.

10. The method of any one of claims 7-9, wherein said toll like receptor comprises toll like receptor 3.

11. The method of claim 10, wherein said activator of toll like receptor 3 is selected from the group consisting of a) Poly IC; b) ARNAX; c) double-stranded RNA; and d) a combination thereof.

12. The method of any one of claims 7-11, wherein said toll like receptor is TLR-4.

13. The method of claim 12, wherein said activator of TLR-4 is LPS, Buprenorphine, Carbamazepine, Fentanyl, Levorphanol, Methadone, Cocaine, Morphine, Oxcarbazepine, Oxycodone, Pethidine, Glucuronoxylomannan from Cryptococcus, Morphine-3-glucuronide, lipoteichoic acid, β-defensin 2, small molecular weight hyaluronic acid, fibronectin EDA, snapin, tenascin C, or a combination thereof.

14. The method of any one of claims 7-13, wherein said toll like receptor is TLR-5.

15. The method of claim 14, wherein said activator of TLR-5 is flagellin.

16. The method of any one of claims 7-15, wherein said toll like receptor is TLR-6.

17. The method of claim 16, wherein said activator of TLR-6 is FSL-1.

18. The method of any one of claims 7-17, wherein said toll like receptor is TLR-7.

19. The method of claim 18, wherein said activator of TLR-7 is imiquimod.

20. The method of any one of claims 7-19, wherein said toll like receptor is TLR-8.

21. The method of claim 20, wherein said activator of TLR8 is ssRNA40/LyoVec.

22. The method of any one of claims 7-21, wherein said toll like receptor is TLR-9.

23. The method of claim 22, wherein said activator of TLR-9 is a CpG oligonucleotide.

24. The method of claim 22, wherein said activator of TLR-9 is ODN2006.

25. The method of claim 2, wherein said activator of TLR-9 is Agatolimod.

26. The method of any one of claims 1-25, wherein said adjuvant is Chloroquine and/or hydroxychloroquine or a functionally active derivative thereof.

27. The method of claim 26, wherein said hydroxychloroquine is administered at a concentration and frequency sufficient to reduce viral replication.

28. The method of claim 26 or 27, wherein said hydroxychloroquine is administered at a concentration and frequency sufficient to reduce activation of TLR-9.

29. The method of any one of claims 26-28, wherein said hydroxychloroquine is administered at a concentration and frequency sufficient to protect pulmonary type 2 epithelial cells.

30. The method of any one of claims 26-29, wherein said hydroxychloroquine is administered at a concentration and frequency sufficient to reduce production of one or more inflammatory cytokines in the lung.

31. The method of claim 30, wherein said inflammatory cytokine is selected from the group consisting of: a) interleukin-1; b) interleukin-6; c) interleukin-8; d) interleukin-11; e) interleukin-15; interleukin-17; h) interleukin-18; i) interleukin-23; j) TNF-alpha; k) angiopoietin; l) HMGB-1; and m) a combination thereof.

32. The method of any one of claims 1-31, wherein said adjuvant is resveratrol or a functionally active derivative thereof.

33. The method of any one of claims 1-32, wherein said adjuvant is losartan or a functionally active derivative thereof.

34. The method of any one of claims 1-33, wherein said adjuvant is azithromycin or a functionally active derivative thereof.

35. The method of any one of claims 1-34, wherein said fibroblasts are either allogeneic, autologous or syngeneic to the recipient.

36. The method of any one of claims 1-35, wherein said fibroblasts are exposed to low level laser irradiation prior to administration.

37. The method of any one of claims 1-36, wherein said fibroblasts are derived from a source of tissues selected from the group consisting of a) dermal; b) placental; c) hair follicle; d) deciduous tooth; e) omentum; f) placenta; g) Wharton's jelly; h) bone marrow; i) adipose tissue; j) amniotic membrane; k) amniotic fluid; l) peripheral blood; and m) a combination thereof.

38. The method of claim 37, wherein said peripheral blood is mobilized to enhance concentration of fibroblasts before isolation of fibroblasts.

39. The method of claim 38, wherein said mobilization is achieved by treatment of the individual with one or more agents selected from the group consisting of a) G-CSF; b) M-CSF; c) GM-CSF; d) Mozibil; e) flt-3 ligand; and f) a combination thereof.

40. The method of any one of claims 1-39, wherein said fibroblasts express CD73.

41. The method of any one of claims 1-40, wherein said ARDS is caused by one or more factors selected from the group consisting of a) cytokine storm; b) immunological cell infiltration; c) bacterial infection; d) viral infection; e) systemic inflammatory response syndrome; f) systemic inflammation; g) acute radiation syndrome; h) sepsis; and i) a combination thereof.

42. The method of any one of claims 1-41, wherein said fibroblasts are administered intravenously, intranasally, intratracheally, or a combination thereof.

43. The method of any one of claims 1-42, wherein said fibroblasts are pre-activated with one or more agents capable of enhancing fibroblast therapeutic activity.

44. The method of claim 43, wherein said fibroblast therapeutic activity is selected from the group consisting of a) mobility towards a chemotactic agent; b) production of anti-inflammatory agents; c) production of anti-apoptotic agents and d) a combination thereof.

45. The method of claim 44, wherein said mobility towards a chemotactic agent is mediated by enhanced expression of a receptor associated with enhanced chemotaxis.

46. The method of claim 45, wherein said receptor associated with enhanced chemotaxis is CXCR4.

47. The method of claim 44, wherein said anti-inflammatory factors are selected from the group consisting of a) IL-4; b) IL-10; c) IL-13; d) IL-20; e) IL-27; f) IL-35; g) PGE-2; h) indolamine 2,3 deoxygenase; i) TGF-beta; j) EGF; and k) a combination thereof.

48. The method of any one of claims 1-47, wherein said fibroblasts are modified to express an enhanced level of one or more therapeutic cytokines.

49. The method of claim 48, wherein said therapeutic cytokine is selected from the group consisting of: a) one or more cytokines that inhibit apoptosis; b) one or more cytokines that act as growth factors; c) one or more cytokines that act as immune modulators/anti-inflammatory agents; and d) a combination thereof.

50. The method of claim 49, wherein said cytokines that inhibit apoptosis are selected from the group consisting of a) EGF; b) VEGF; c) angiopoietin; and d) a combination thereof.

51. The method of claim 49, wherein said cytokines that act as growth factors are selected from the group consisting of a) HGF; b) FGF-1; c) FGF-2; d) KGF; e) CTNF; and f) a combination thereof.

52. The method of claim 49, wherein said cytokines that act as immune modulators/anti-inflammatory agents are selected from the group consisting of a) IL-4; b) IL-10; c) IL-13; d) IL-20; e) IL-27; f) IL-35; g) PGE-2; h) indolamine 2,3 deoxygenase; i) TGF-beta; j) neuroaminidase; and k) a combination thereof.

53. A method of reprogramming monocytes in the lung of an individual suffering from or having a risk for ARDS, comprising administering to the individual an effective amount of one or more adjuvants with a therapeutically effective amount of fibroblasts and/or fibroblast-derived products, wherein said adjuvant comprises one or more peptides, one or more activators of one or more toll like receptors, chloroquine and/or hydroxychloroquine, resveratrol, losartan, azithromycin, or a mixture thereof.

54. The method of claim 53, wherein said fibroblasts are derived from a source of tissues selected from the group consisting of a) dermal; b) placental; c) hair follicle; d) deciduous tooth; e) omentum; f) placenta; g) Wharton's jelly; h) bone marrow; i) adipose tissue; j) amniotic membrane; k) amniotic fluid; l) peripheral blood; and m) a combination thereof.

55. The method of claim 54, wherein said peripheral blood is mobilized to enhance concentration of fibroblasts before isolation of fibroblasts.

56. The method of claim 55, wherein said mobilization is achieved by treatment of said individual with one or more agents selected from the group consisting of a) G-CSF; b) M-CSF; c) GM-CSF; d) Mozibil; e) flt-3 ligand; and f) a combination thereof.

57. The method of any one of claims 53-56, wherein said fibroblast is a) allogeneic; b) autologous; or c) xenogeneic with respect to the individual.

58. The method of any one of claims 53-57, wherein said ARDS is caused by one or more factors selected from the group consisting of a) cytokine storm; b) immunological cell infiltration; c) bacterial infection; d) viral infection; e) systemic inflammatory response syndrome; f) systemic inflammation; g) acute radiation syndrome; h) sepsis; and i) a combination thereof.

59. The method of any one of claims 53-58, wherein said fibroblasts are administered intravenously, intranasally, and/or intratracheally to the individual.

60. The method of any one of claims 53-59, wherein said fibroblasts are pre-activated with one or more agents capable of enhancing fibroblast therapeutic activity.

61. The method of claim 60, wherein said fibroblast therapeutic activity is selected from the group consisting of a) mobility towards one or more chemotactic agents; b) production of one or more anti-inflammatory agents; c) production of one or more anti-apoptotic agents; and d) a combination thereof.

62. The method of claim 61, wherein said mobility towards a chemotactic agent is mediated by enhanced expression of one or more receptors associated with enhanced chemotaxis.

63. The method of claim 62, wherein said receptor associated with enhanced chemotaxis is CXCR4.

64. The method of claim 61, wherein said anti-inflammatory factors are selected from the group consisting of a) IL-4; b) IL-10; c) IL-13; d) IL-20; e) IL-27; f) IL-35; g) PGE-2; h) indolamine 2,3 deoxygenase; i) TGF-beta; j) EGF; and k) a combination thereof.

65. The method of any one of claims 1-64, wherein said fibroblasts are modified to express enhanced levels of one or more therapeutic cytokines.

66. The method of claim 65, wherein said therapeutic cytokines are selected from the group consisting of a) cytokines that inhibit apoptosis; b) cytokines that act as growth factors; c) cytokines that act as immune modulators/anti-inflammatory agents; and d) a combination thereof.

67. The method of claim 66, wherein said cytokines that inhibit apoptosis are selected from the group consisting of a) EGF; b) VEGF; c) angiopoietin; and; d) a combination thereof.

68. The method of claim 66, wherein said cytokines that act as growth factors are selected from the group consisting of a) HGF; b) FGF-1; c) FGF-2; d) KGF; e) CTNF; and f) a combination thereof.

69. The method of claim 66, wherein said cytokines that act as immune modulators/anti-inflammatory agents are selected from the group consisting of a) IL-4; b) IL-10; c) IL-13; d) IL-20; e) IL-27; f) IL-35; g) PGE-2; h) indolamine 2,3 deoxygenase; i) TGF-beta; j) neuroaminidase; and k) a combination thereof.

70. The method of any one of claims 1-69, wherein the individual is administered one or more additional therapies.

71. The method of claim 70, wherein the additional therapy is administered sequentially or simultaneously with the fibroblasts and/or one or more adjuvants.

72. The method of claim 70 or 71, wherein the additional therapy is ventilation, a glucocorticoid, a surfactant, inhaled nitric oxide, an antioxidant, a protease inhibitor, a recombinant human activated protein C, a.beta.2-agonist, lisofylline, a statin, inhaled heparin, a diuretic, a sedative, an analgesic, a muscle relaxant, an antibiotic, inhaled prostacyclin, inhaled synthetic prostacydin analog, ketoconazole, alprostadil, keratinocyte growth factor, beta-agonists, human monoclonal antibody (mAb) against tissue factor VIIa (TS factor 7a), interferon receptor agonists, insulin, perfluorocarbon, budesonide, recombinant human angiotensin-converting enzyme (ACE), recombinant human Clara cell 10 kDa (CC10) protein, tissue plasminogen activator, human mesenchymal stem cells, nutritional therapy, methylprednisolone, dexamethasone, prednisone, prednisolone, betamethasone, triamcinolone, triamcinolone acetonide, beclometasone, albuterol, lisofylline, rosuvastatin, inhaled heparin, inhaled nitric oxide, recombinant human activated protein C, ibuprofen, naproxen, acetaminophen, cisatracurium besylate, procysteine, acetylcysteine, inhaled prostacydin, ketoconazole, alprostadil, keratinocyte growth factor, beta-agonists, human monoclonal antibody (mAb) against tissue factor VIIa (TS factor 7a), insulin, perfluorocarbon, budesonide, recombinant human angiotensin-converting enzyme (ACE), recombinant human Clara cell 10 kDa (CC10) protein, tissue plasminogen activator, human mesenchymal stem cells, a nutritional therapy, a combination of omega-3 fatty acids, antioxidants, .gamma.-linolenic acids with isocaloric foods, mechanical ventilation, or a combination thereof.

73. The method of any one of claims 1-72, wherein said fibroblasts are endowed with ability to suppress viral infection.

74. The method of claim 73, wherein said suppression of viral infection is achieved through production of interferon.

75. The method of claim 93, wherein said interferon is selected from the group consisting of a) interferon alpha; b) interferon beta; c) interferon gamma; d) interferon tau; e) interferon omega; and f) a combination thereof.

76. The method of any one of claims 1-75, wherein said fibroblast-derived products comprise exosomes.

77. The method of claim 76, wherein the exosomes express CD8.

78. The method of claim 76 or 77, wherein said exosomes express annexin-V.


SEQUENCE LISTING (5 sequences)

SEQ ID NO: 1 - protein, 15 aa, Artificial Sequence
  Description of Artificial Sequence Synthetic peptide
GEPPPGKPADDAGLV

SEQ ID NO: 2 - protein, 43 aa, Artificial Sequence
  Description of Artificial Sequence Synthetic polypeptide
SDKPDMAEIEKFDKSKLKKTETQEKNPLPSKETIEQEKQAGES

SEQ ID NO: 3 - protein, 6 aa, Artificial Sequence
  Description of Artificial Sequence Synthetic peptide
CSKKKK

SEQ ID NO: 4 - protein, 6 aa, Artificial Sequence
  Description of Artificial Sequence Synthetic peptide
CSKKKK

SEQ ID NO: 5 - protein, 6 aa, Artificial Sequence
  Description of Artificial Sequence Synthetic peptide
CSKKKK